(19) United States
(12) Patent Application Publication
(10) Pub. No.: US 2021/0322537 A1
(43) Pub. Date: Oct. 21, 2021

(54) Method For Preparation of Quick Dissolving Thin Films Containing Bioactive Material With Enhanced Thermal Stability

(71) Applicant: Aridis Pharmaceuticals, Inc., San Jose, CA (US)

(72) Inventors: Van Nguyen, San Jose, CA (US); Phillip M. Lovalenti, Mountain View, CA (US); Vu Truong-Le, Campbell, CA (US); Jeff Anderl, Pacifica, CA (US); Satoshi Ohtake, Chesterfield, MO (US); Atul Saxena, Milpitas, CA (US)

(21) Appl. No.: 16/307,314

(22) PCT Filed: Jun. 6, 2017
(86) PCT No.: PCT/US2017/036207

§ 371 Date: Feb. 12, 2020

Related U.S. Application Data
(60) Provisional application No. 62/347,009, filed on Jun. 7, 2016.

(51) Int. Cl. A61K 39/15; A61K 9/70; A61P 31/14; A61K 9/00; A61K 47/32; A61K 47/10; A61K 39/112; A61K 47/02

(52) U.S. Cl. CPC A61K 39/15; A61K 9/7007; A61P 31/14; A61K 47/02; A61K 47/32; A61K 47/10; A61K 39/0275; A61K 9/006

(57) ABSTRACT

Methods for the preparation of polymeric films which encase and preserve bioactive agents. In particular, the invention is directed to the preparation of oral thin films containing bioactive proteins or viruses. For example, methods and compositions are disclosed for preservation of rotavirus and antibodies in thin dry films.

### FIELD OF THE INVENTION

In particular, the invention is directed to the preparation of oral thin films. The invention is directed to methods and compositions for preparation of thin films for delivery of bioactive materials by the oral route. The thin films provide process stability, thermal stability, and storage stability for a variety of bioactive materials. The bioactive agent, such as a vaccine or antibody, e.g., in the form of a solution or dried powder, is mixed with a polymer matrix, then dried into a thin film with good long term stability.

### BACKGROUND OF THE INVENTION

Oral thin films (OTF's) have been identified as alternative dosage presentations to the widely used tablets and liquid drops. The advantages of this delivery format include accurate dosing, small packaging size, easy handling and administration, patient compliance and/or acceptance and simple, cost effective manufacturing processes complementary with current industry practices. Oral delivery thin-film strips are designed to wet and dissolve quickly upon contact with saliva and buccal tissue, releasing the contained pharmaceutical product. The main components of oral thin films are typically one or more hydrophilic polymers, some of which have good mucoadhesive properties. In such case, the polymeric thin film strongly adheres to buccal tissue until complete dissolution. Quick dissolution and mucoadhesion are key properties important for patient compliance and improved administration of the contained therapeutics.

Breath fresheners such as Listerine® have been encased in oral thin films and sold commercially, but recently more complex products such as over-the-counter medications, including dental care and flu medicine have been successfully encased in oral thin films, in addition to several prescription small molecule medications such as Suboxone®, Zuplenz®, ONSOLIS® or BUNAVAIL®. However, the processes to create these oral thin films are generally not designed to encase the large, more thermally labile bioactives such as proteins, live-attenuated viruses and bacterial vaccines. Commercial film manufacturing processes typically require high temperatures, potentially inactivating solvents or other extreme conditions that could denature potential biotherapeutic agents leading to significant loss in potency and, as a consequence, their bioactivity.

Drugs delivered through the gastrointestinal (GI) tract are subjected to low pH (high acidity) and harsh enzymatic environment in the gastric cavity. Protein drugs, nucleic acids and vaccines are not resistant to these conditions, and are denatured and degraded, leading to significant loss in their bioactivity.

When incorporating bioactive components into the thin films, care must be taken to develop a process that preserves bioactivity. Further, in order to maintain that bioactivity for the shelf life of the product, key excipients are needed in developing a thin film formulation to ensure potency preservation over time under the intended storage conditions.

In view of the above, a need exists for compositions that can deliver bioactive materials more efficiently. We believe it would be desirable to have OTFs that are adapted to deliver a wide range of bioactive agents, e.g., in an efficient manner. Benefits could also be realized if the OTFs were designed to provide shelf life commensurate with other delivery systems and compositions. The present invention provides these and other features that will be apparent upon review of the following.

### SUMMARY OF THE INVENTION

The inventions are directed to methods for preparation of quick dissolving thin films containing bioactive material while providing enhanced stability in the manufacturing process and storage. The compositions contain the bioactive agent, excipients, and matrix polymers that work together to provide a stable efficient delivery system. The methods include the steps of blending the bioactive agent, excipients, and polymer to form a wet blend. The wet blend is applied to a flat surface for drying, using heat and/or vacuum conditions, to form a thin film. The excipients and polymers are selected, as described herein, to provide high process recoveries, long shelf life, and good dissolution time. As a general rule, the formula constituents are balanced to provide low molecular motion, retained moisture of between about 10% and 1.5%, and a protective but water soluble polymer matrix.

To protect the bioactive agent through the harsh thin film manufacturing conditions, unique combinations of pharmaceutical excipients and drying process technologies were developed. Maintaining storage stability and simplifying the distribution and administration procedures are critical in order to implement large scale therapeutic and prophylactic treatments. Accordingly, the methods of the present invention include the fabrication of a polymeric film which comprises bioactive materials including proteins and vaccines that are stabilized with unique pharmaceutical excipient combinations. A range of formulations with a variety of excipients and polymer compositions, in various solvent systems, were presented in order to prepare films that preserve bioactivity through both fabrication and during elevated temperature storage. Different solvent evaporation techniques were also developed for the formation of these films. Preferred embodiments of this invention teach oral thin films and manufacturing methods using polymers in combination with pharmaceutical, excipient-stabilized bioactive agents in the presence of a buffer.

In one embodiment of the film, the biologic agent is a rotavirus (e.g., an attenuated rotavirus vaccine). The composition of the thin dry film includes stabilizing excipients and a polymer matrix. The stabilizing excipients can include buffers, polymers, plasticizers, divalent cations, surfactants, sugars, and/or solvents, which aid in processing and enhance the viability of the rotavirus during processing and in storage. In certain embodiments, the composition comprises rotavirus formulated in any of F1 to F8 (see, e.g., Table 1 of Example 2, below) excipient solution formulations and their near equivalents (each component present within 25% of identified values). The stock solutions of bioactive material and excipient solution are mixed with a matrix polymer (e.g., polyvinyl alcohol (PVA) and/or polyethylene oxide (PEO)) to provide a wet film blend ready to process into a dry film, e.g., according to methods described herein. In a more preferred embodiment, the rotavirus is formulated with any of F1 to F3 excipient solutions and blended into a wet film blend with PVA. We find a specific rotavirus composition with outstanding stability and handling characteristics can be prepared using the F1 excipient formulation (potassium phosphate, citric acid, sucrose, sorbitol, calcium chloride, zinc chloride, and gelatin) using PVA as the matrix polymer, e.g., dried to a flat film in a convection oven for 1-2 hours at 60° C.

The rotavirus thin film can have certain desirable characteristics. For example, the composition can be formed into a thin film having a residual moisture of from 2% to 7%; the rotavirus can be present in a titer expressed as fluorescent focus unit (ffu) per milligram (mg) of dried film between 4 log ffu/100 mg to 7 log ffu/100 mg, or about 6 log ffu/100 mg. The film can be dried by exposure to 45° C. to 80° C. (or 50° C. to 65° C.) for 0.5 to 3 hours. The film can have a major plane with a thickness (through the dimension perpendicular to the plane) ranging from 20 microns to 400 microns. The matrix polymer can be at least 4-fold more than any plasticizers in the formulation. The composition can beneficially be prepared from an excipient formulation containing at least 1 wt % sorbitol. The composition can include any of rotavirus strains, particularly strains G1, G2, G3 and/or G4.

The thin films can also incorporate bioactive proteins, e.g., such as antibodies. For example, a thin film composition with a protein active agent (such as a monoclonal antibody) can include the protein in an excipient solution formulation blended with a matrix polymer, dried to a thin film. In certain embodiments, the composition comprises an antibody formulated in any of M3, M5, M6, or M7 (see, e.g., Table 20 of Example 21, below) stabilizer formulations and their near equivalents (each component present within 25% of identified values). The embodiments are blended into a wet film blend with a polymer (e.g., poloxamer, polyvinyl alcohol (PVA) and/or polyethylene oxide (PEO)) for processing into a dry film, e.g., according to methods described herein.

The methods of producing the thin films can include blending a solution or suspension of the bioactive agent, excipients, and polymer, to provide a wet blend. The wet blend can be dried on a surface under ambient conditions, with added heat, or under “vacuum” conditions (e.g., freeze drying or vacuum drying). For example, the wet blend can be spread onto a planar surface and exposed to air currents and/or heat (e.g., 15 minutes to 4 hours at 30° C. to 70° C.) until the residual moisture of the thin film product ranges from about 1.5% to 10%. Thin films typically have a thickness, perpendicular to the major plane, of about 50 microns to about 500 microns.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a surface” can include a combination of two or more surfaces; reference to “bacteria” can include mixtures of bacteria, and the like.

Unless defined otherwise, all technical and scientific terms used herein have the same meaning as commonly understood by one of ordinary skill in the art to which the invention pertains. Before describing the present invention in detail, it is to be understood that this invention is not limited to examples that are disclosed, for example, to the bioactive agents, polymers, excipients, vaccines, or concentration ranges, and the like. It is also to be understood that the terminology used herein is for the purpose of describing particular embodiments only, and is not intended to be limiting.

Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

The term “about”, as used herein, indicates the value of a given quantity can include quantities ranging within 10% of the stated value, or optionally within 5% of the value, or in some embodiments within 1% of the value.

“Pharmaceutically acceptable” refers to those active agents, salts, and excipients which are, within the scope of sound medical judgment, suitable for use in contact with the tissues or humans and lower animals without undue toxicity, irritation, allergic response and the like, commensurate with a reasonable benefit/risk ratio, and effective for their intended use. Pharmaceutically acceptable excipients (vehicles, additives) are those which can reasonably be administered to a subject mammal to provide an effective dose of the active ingredient employed. Preferably, these are excipients which the Federal Drug Administration (FDA) have to date designated as ‘Generally Regarded as Safe’ (GRAS).

A “polyol” is as known in the art, e.g., molecules with multiple hydroxyl groups, and includes, e.g., sugars (reducing and nonreducing sugars), sugar alcohols, and sugar acids. Preferred polyols herein have a molecular weight which is less than about 600 kDa (e.g. in the range from about 120 to about 400 kDa). A “reducing sugar” is a polyol which contains a hemiacetal group that can reduce metal ions or react covalently with lysine and other amino groups in proteins. A “nonreducing sugar” is a sugar which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof.

The term “thin film” is as would be understood in common usage and by one of skill in the art on reading this specification. For example, a thin film can be a thin sheet of material having a thickness dimension markedly less than the dimension across the major plane of the sheet (e.g., a thickness less than 1% the sheet length or width at the end of drying). For example, in a typical embodiment as a quick dissolving carrier of a bioactive agent, a thin film is typically a sheet having a thickness of less than about 1 mm, 0.25 mm, 0.1 mm, 0.05 mm or less.

The term “wet blend” refers to a combination of a bioactive agent, excipient solution, and matrix polymer, as described herein. The wet blend is formulated to feed into drying processes on a surface, e.g., where most of the water is removed to result in a dry thin film.

The term “matrix polymer”, as used herein, refers to the major one or two polymers in the wet blend (or in the matrix polymer stock) that provide a polymer matrix to the dried thin films. The term is not intended to refer to all polymers, but typically those dissolved or suspended in the matrix polymer stock that is combined with the bioactive stock solution to provide the wet blend. Polymers specifically excluded as matrix polymers of the present films are natural proteins, nucleic acids, and starches. Exemplary matrix polymers in the thin films include, e.g., polyethylene oxide (PEO), and polyvinyl alcohol (PVA), and polyvinyl pyrrolidone.

The term “plasticizer” refers to an excipient compound that lowers the glass transition temperature of a solidified glassy matrix. Here, the plasticizer is included in the wet blend as a dissolved solid and serves to modify the physical properties of the dried thin film imparting it with desirable functionality at the appropriate concentration. Exemplary plasticizers in the thin films include, e.g., glycerol and sorbitol.

### BRIEF DESCRIPTION OF THE DRAWINGS

FIG. 1 shows a flow diagram of a typical process of preparing a bioactive dry thin film.

FIG. 2 shows storage stability of quadrivalent OTF formulation F2 at a) 4° C., b) 25° C. and c) 40° C.

FIG. 3 shows storage stability of quadrivalent OTF formulation F3 at a) 4° C., b) 25° C., and c) 40° C.

FIG. 4 shows stability of various OTF formulations containing CaCO₃ dispersed solids. FIG. 4A—45° C. stability of various 5% Sucrose OTF formulations containing CaCO₃ dispersed solids. Note: moisture content levels are labeled to the top of the t0 bars. FIG. 4B—45° C. stability of various 10% Sucrose/50 mM KPO₄ OTF formulations containing CaCO₃ dispersed solids. Note: moisture content levels are labeled to the top of the t0 bars. FIG. 4C—45° C. stability of various 20% Sucrose OTF formulations containing CaCO₃ dispersed solids. Note: moisture content levels are labeled to the top of the t0 bars.

FIG. 5 shows stool anti-RRV IgA response in 7-day old mouse pups exposed to different dosage forms of RRV. Stool samples were not able to be retrieved on week 2 for Group 2.

FIG. 6 shows serum anti-RRV IgG response in 7-day old mouse pups exposed to different dosage forms of RRV. Serum samples were not able to be collected until week 4.

### DETAILED DESCRIPTION

The present invention is directed to thin film compositions incorporating bioactive agents and configured to provide efficient delivery and long term stability of the agent. The thin film polymer compositions are low in residual moisture and reduce exposure of the biologic agent to destabilizing phenomenon such as heat, light, oxidation, and moisture. The inventions include methods of preparing thin dry films incorporating bioactive materials.

Initial studies (see, e.g., Example 26) have shown that oral administration with dry thin films can provide efficacy comparable to liquid dosage forms. Further work, e.g., in Examples 2-25 below, has identified formulations and processes to incorporate various bioactive agents into the films with high process recovery, extended shelf life, and good dosage bioavailability on administration.

In one embodiment, bioactive material in the form of a live virus vaccine is stabilized in solution containing sugar, buffer, and divalent cations, then added to the polymer matrix mixture and then dried to form a thin stable film. In another embodiment, the vaccine is stabilized in sugar, buffer, and divalent cations as a dry powder then added to the polymer mixture to form a thin film. Both the liquid and dry powder formulations can further contain additional components, including a surfactant, polymer, amino acids, and antacids.

Other bioactive agents, such as nucleic acids and proteins can also be stabilized using the compositions and methods for making thin films. For example, antibodies can be formulated into specialized excipient solutions, and then blended with matrix polymers for drying into films. The antibody, encased in the matrix with high process recovery, shows remarkable stability in storage and bioavailability on administration.

### I. Formulations and Product Intermediates for Preparation of Bioactive Thin Films

For the preparation of a dry thin film comprising one or more bioactive agents, a bioactive material sample is typically mixed with an excipient solution to prepare a bioactive stock solution or suspension (bioactive stock solution). The bioactive stock solution is blended with a matrix polymer (or a mix of matrix polymers) to prepare a wet blend for drying on a surface to form a dry thin film incorporating the bioactive agent.

### Stock Solutions or Suspensions of Bioactive Agents

Stock solutions include the bioactive agent in an aqueous solution (e.g., antibodies) or suspension (e.g., viruses) along with excipients that provide a stable environment during processing. Many of the excipients in the stock solution also play a roll in extending shelf life of the bioactive agents in the dried thin film.

The bioactive agents for incorporation into thin films can include, e.g., bacteria, viruses, proteins, nucleic acids, and small molecule pharmaceuticals. For example, the bioactive agents can include viral vaccine, a bacterial vaccine, a nucleic acid, a protein, an antibody, an enzyme, a growth factor, a cytokine, an adjuvant, or a virus-like particle.

The bioactive agent is often initially available in a relatively purified solution or suspension. For example, the bioactive agent can be the final product of purification or concentration process. This bioactive product is combined with an excipient solution to prepare a stock solution intended for blending with polymer. The bioactive agent could be dialyzed into the excipient solution, but it is often convenient to simply blend the agent into an excipient solution (e.g., one part purified agent solution with 4 parts thin film excipient solution) to form the bioactive stock solution. Alternately, e.g., where the bioactive agent is received in a freeze dried or spray dried form, the agent can be simply reconstituted in the excipient solution to make the bioactive stock solution.

The bioactive stock solution is then blended with a matrix polymer, or matrix polymer mixture, to provide a wet blend for thin film drying. Alternately, the excipient solution and matrix polymer(s) can be mixed before addition of the bioactive agent solution or suspension, forming the wet blend

### Excipient Solutions

Bioactive agents are combined with excipient solution formulations to stabilize the bioactive agent during processing, and to provide a stable environment for extended storage of the active thin film. Exemplary excipient solutions are presented in Table 1 of Example 2, Table 20 of Example 21, and the bacterial excipient formulations of Example 22, below.

Formulations F1 to F24 of Table 1 have been found useful for the processing and stability of viruses, in the thin films of the invention. The virus excipient solutions can include, e.g. buffers, polyols (such as sugars), plasticizers, salts, and/or gelatin.

In preferred formulations for viruses, the excipient solution includes potassium phosphate, citrate, sucrose, sorbitol, calcium ions, zinc ions, and gelatin. In more preferred embodiments, the formulations include the sorbitol at about a 1.6 wt %. These formulations work particularly well in combination with the PVA matrix polymer. These formulations are well adapted for processing and storage of rotavirus in thin films.

Formulations M1 to M7 of Table 20 have been found useful for the processing and stability of protein bioactive agents, in the thin films of the invention. The protein agent excipient solutions can include, e.g., buffers, sugars, polyols, and/or polymers. Note that polymers in the stock solutions are not considered “matrix polymers” of the thin films, unless they meet the requirements outlined below in the Matrix Polymer section. For example, an antibody protein is not considered the matrix polymer in a film configured to protect the antibody.

In preferred formulations for proteins, the excipient solution includes histidine, sucrose, sorbitol, and polysorbate. The triblock copolymer poloxamer 188 can provide additional benefits. These formulations work particularly well in combination with the PVA matrix polymer. These formulations are well adapted to instances where the bioactive agent protein is an antibody.

With regard to bacteria, a good functional excipient solution can include, e.g., potassium phosphate buffer, trehalose, methionine, and gelatin. For example, the T2 formulation was composed of 25% trehalose, 1% methionine, 5% gelatin, and 25 mM potassium phosphate at pH 8. Alternately, the bacterial excipient stock can simply include a buffer, e.g., for hardy bacteria, such as many Enterobacteriaceae.

The total solids percent of excipient solutions is generally fairly high, e.g., to minimize drying times of the wet blend process intermediate. For example, total solids in the excipient solutions can range from less than about 5 wt % to more than 50 wt %, from 10% to 35%, from 15% to 30%, or about 25%. The bulk of the excipient solids are usually some form of sugar(s) and/or other polyol(s), e.g., acting as a fast dissolving bulking agents and stabilizers.

Buffers can be included in the excipient solutions of the invention to provide a favorable environment for formulation constituents' solubility, and to enhance stability of the bioactive agent. Typical buffers of the invention are, e.g., potassium phosphate, sodium phosphate, sodium acetate, citrate, sodium succinate, histidine, imidazole, ammonium bicarbonate, a carbonate, HEPES, tris, tartarate, maleate, lactate, magnesium oxide, aluminum oxide, aluminum hydroxide with magnesium hydroxide, aluminum carbonate gel, sodium bicarbonate, hydrotalcite, sucralfate, and bismuth subsalicylate. pH levels can be adjusted in the formulations, compositions, and reconstituted products of the invention, e.g., to a pH ranging from about pH 4 to about pH 10, from about pH 6 to about pH 8, and, more typically, near neutral or about pH 7.2.

With regard to rotavirus, it is desirable to maintain the pH in a range from pH 5 to 7. For stability of live rotavirus, a pH range of 6.0 to 6.5 is desirable. A preferred pH to enhance stability of Rotavirus capsids is about pH 6.3.

Viruses and proteins are typically more stable in the presence of substantial amounts of polyol, such as a substantially water soluble sugar. In preferred embodiments, the formulation sugar is a monosaccharide or disaccharide. In one aspect, the sugar is present in the excipient solution in an amount ranging from less than about 5% to 60%, 10% and 35%, 15% and 25%, or about 20% by weight. In preferred embodiments the sugars are present in the excipient solutions at a concentration ranging from about 20% to about 30% by weight. More preferred sugars include, e.g., sucrose, mannitol, lactose, dextrose, fucose, trehalose, polyaspartic acid, inositol hexaphosphate (phytic acid), sialic acid and N-acetylneuraminic acid-lactose. In a typical embodiment, the sugar is trehalose or sucrose. Polyols of the excipient solutions can include, e.g., non-reducing sugars, reducing sugars, sugar alcohols and sugar acids. Polyols can include, e.g., sucrose, trehalose, sorbose, melezitose, stachyose, raffinose, fructose, mannose, maltose, lactose, arabinose, xylose, ribose, rhamnose, galactose and glucose, mannitol, xylitol, erythritol, threitol, L-gluconate, and/or the like.

Zwitterions can help stabilize protein structures and contribute to pH buffering. In some embodiments of the invention amino acids are present in the excipient solution in amounts ranging from about 0 mM to 20 mM, or about 10 mM. Preferred amino acids for incorporation into the inventive formulations are, e.g., histidine, arginine, lysine, methionine, serine, glutamic acid, and/or the like. In a most preferred embodiment, the amino acid is histidine at about 10 mM.

Surfactants can be present in the excipient solutions, e.g., to stabilize and enhance the solubility of other constituents. Surfactants of the formulations and compositions can include, e.g., polyethylene glycol, polypropylene glycol, polyethylene glycol/polypropylene glycol block copolymers, polyethylene glycol alkyl ethers, polypropylene glycol alkyl ethers, polyethylene glycol/polypropylene glycol ether block copolymers, alkylarylsulfonates, phenylsulfonates, alkyl sulfates, alkyl sulfonates, alkyl ether sulfates, alkyl aryl ether sulfates, alkyl polyglycol ether phosphates, polyaryl phenyl ether phosphates, alkylsulfosuccinates, olefin sulfonates, paraffin sulfonates, petroleum sulfonates, taurides, sarcosides, fatty acids, alkylnaphthalenesulfonic acids, naphthalenesulfonic acids, lignosulfonic acids, condensates of sulfonated naphthalenes with formaldehyde, or condensates of sulfonated naphthalenes with formaldehyde and phenol, lignin-sulfite waste liquor, alkyl phosphates, quaternary ammonium compounds, amine oxides, betaines, and/or the like. Tween® and Pleuronic® surfactants, such as, e.g., polyethylene glycol sorbitan monolaurate, polyoxyethylenesorbitan monooleate, or block copolymers of polyethylene and polypropylene glycol, are particularly preferred surfactants of the invention. In preferred embodiments, the surfactant is a non-ionic surfactant such as a polysorbate, a polyoxyethylene alkyl ether, a nonaethylene glycol octylphenyl ether, a hepatethylene glycol octylphenyl ether, a sorbitan trioleate, and a polyoxyethylene-polyoxypropylene block copolymer. Surfactants (if present) can be present in formulations of the invention in amounts of, e.g., about 0.01 weight percent to about 1 weight percent.

Divalent cations can help stabilize proteins and viruses in solution and in the dry thin film. Particularly, with respect to rotavirus embodiments, it can be desirable that the Zn²⁺ and/or Ca²⁺ be present in the excipient solution at a concentration of at least 0.5 mM. It is preferred that Zn²⁺ be present at a concentration ranging from about 1 mM to about 20 mM, from about 2 mM to about 10 mM, from about 3 mM to about 6 mM zinc ions, or about 4 mM zinc ions. For some formulations, particularly for certain storage conditions, it can be beneficial to have a combination of both Zn²⁺ and Ca²⁺ ions in the excipient solution.

In certain cases, some plasticizer constituents can be helpful in storage stabilization of the bioactive agent and allowing the dry film to be less brittle for handling on process and administration. Further, some plasticizer can allow retention of less water, for better stability, without the film losing flexibility. A plasticizer that interacts well with the glassy matrix of the film can be sorbitol. It can be desirable that plasticizer be present in the excipient solution for rotavirus at a concentration less than 25% by weight. It is preferred that sorbitol be present at a concentration ranging from about 0 to about 10% by weight, from about 0 to 5% by weight, or about 1.6% by weight.

Flavor ingredients or bar code identifiers can optionally be incorporated into the process materials. Flavors can make the product more appealing to smell or take orally. Bar codes (e.g., nanoparticles or readable nucleic acid sequences) can identify the source of the product batch.

### Matrix Polymers

The present thin films employ a polymer matrix to protect the bioactive agents and to facilitate handling. Matrix polymers of the films are typically not natural polymers. That is, the matrix polymers are not natural nucleic acids, proteins, or starches. Polymers of less than 4 repeat units (tetramer) are not considered matrix polymers of the invention. Gelatin is not considered a matrix polymer of the film, but may be an excipient constituent. Preferred matrix polymers are polyvinyl alcohols (PVA), polyvinyl pyrrolidone, polyethylene oxide, poloxamer, and/or the like. Preferred matrix polymers are generally recognized as safe and ingestible. Preferred matrix polymers are generally more hydrophilic than hydrophobic, and water soluble.

Matrix polymers are typically present in a matrix polymer stock solution at about 25 wt %. The matrix polymer stock solutions can range from less than about 1% to more than about 30 wt %, from 4% to 25%, of about 25% by weight. The total solids in the matrix polymer stock can be lower than in the excipient solutions because it is often more difficult to suspend or dissolve the matrix polymers at high concentrations due to, e.g., solubility, viscosity, and temperature sensitivity issues.

The matrix polymer stock is typically blended with the bioactive stock solution at a ratio ranging from less than about 1:2 (polymer matrix: bioactive stock solution) to more than about 4:1, from 1:1 to 3:1, or about 1:1 for rotavirus formulations and 2:1 for antibody formulations, to prepare a wet blend. With these ratios, the wet blend, and ultimate thin film, can include matrix polymers as a percent of total dissolved solids by weight ranging from less than about 10 wt % to more than 80%, from 30% to 70%, from 40% to 50%, or about 45% matrix polymer. However, for film formulations that include high concentrations of a dispersed antacid, such as calcium carbonate or magnesium oxide powder, ranging up to 50% of total solids, the matrix polymer content indicated here can be cut in half.

### II. Methods of Preparing Bioactive Dry Thin Films

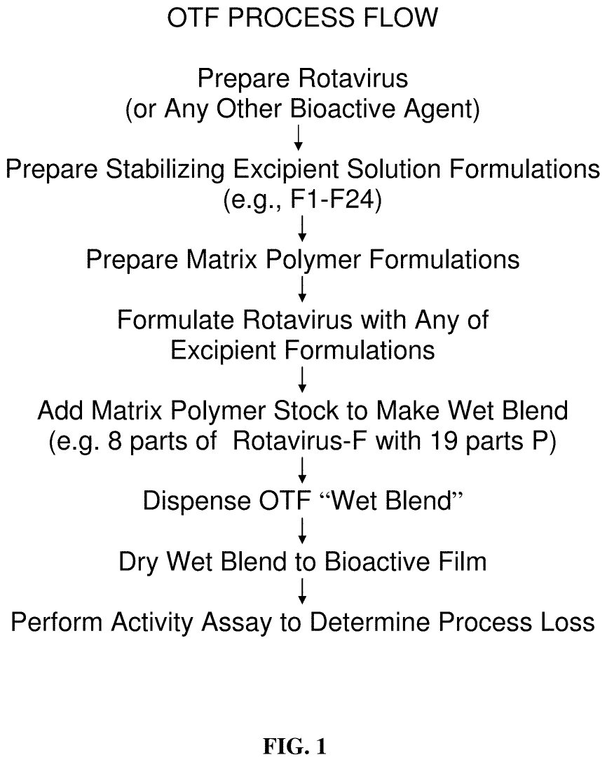

Methods of dry thin film manufacture generally comprise preparation of process solutions, mixture of the solutions, application of the mixture to a surface, drying the mixture, removal film from the drying surface, and storage of the thin film product. See, e.g., the flow diagram of FIG. 1.

The process solutions for manufacturing the films are described above and in the Examples, below. Generally, it is desirable to provide the bioactive solution, excipient solution, and matrix polymer stock with adequate solvent (typically water) for handling, but in minimum amounts, e.g., to reduce drying time and heat stress during the drying step. At various times during processing, it can be useful to degas the product intermediates, to reduce problems of surface denaturation and final product porosity, as well as to maintain surface smoothness and appearance of the final dry film.

There is usually little difficulty in combining the bioactive solution, and excipient solution to prepare the bioactive stock solution. Although the excipient solution can be somewhat viscous due to the high solids (e.g., sugar bulk) component, gentle stirring can usually provide a uniformly dispersed stock solution. Alternately, the bioactive solution can be initially combined with the matrix polymer stock. However, this can often be less desirable due to the higher viscosity (even though typically lower total solids) in the matrix polymer stock, and lack of protective excipients. These issues can vary widely, e.g., depending on the nature of the bioactive agent to be protected.

In some cases, the bioactive agent can be received as a freeze dried cake or powder, or as a spray dried powder. In such cases, the bioactive agent can often be reconstituted directly in the excipient solution. Optionally, when the dried bioactive agent is already formulated with excipient stabilizers, it can be suspended directly in an organic solvent with dissolved matrix polymers to produce the film wet blend.

With the completed combination of the bioactive agent with excipients and matrix polymer, the “wet blend” is ready for application to a surface for drying. The surface is typically a planar surface. The wet blend can be applied and allowed to spread seeking the lowest level by gravity on a level horizontal drying surface. The wet blend can be sprayed or painted, e.g., uniformly onto the drying surface. The surface can alternately not be planar and/or horizontal. For example, the drying surface can be a drum, or the wet blend could be extruded vertically to dry, e.g., as a tape. In any case, it is usually desired to present a large surface relative to volume, to speed drying or allow for less stressful drying conditions.

In one embodiment, the wet blend is applied to a broad planar surface and exposed to heat from above (e.g., warm gas stream and/or IR light) and/or from below with the planar surface itself being heated. In preferred embodiments, the wet blend is dried at a temperature ranging from less than 20° C. to more than about 80° C., from 30° C. to 60° C., or about 50° C. The drying can continue for a time ranging from less than about 0.5 hours to more than about 6 hours, 0.75 hours to 4 hours, or from 1 to 2 hours.

Following exposure to heated drying, additional moisture can be removed from the wet blend by vacuum drying. For many bioactive agents, activity losses in process can be reduced by lower temperature drying. Some porosity may be introduced by vacuum drying, but this will be relatively minor because the wet blend starts out relatively low in water after heated drying. A side benefit of vacuum frying may be faster dissolutions for patient administration. For example, the wet blend can be applied to a surface and exposed to heated drying for 1 to 2 hours at 50° C. Final reduction of residual moisture can then be completed at a low pressure (e.g., 100 mTorr) with a lower temperature, e.g., 4° C. for an adequate time.

The wet blend is usually applied fairly thin, yet drying takes some time, e.g., due to the hydrophilic nature of formulation constituents. This can be mitigated somewhat by including a volatile (e.g., organic) solvent in the wet blend. For example, chloroform, ethanol, heptane, isopropyl alcohol (IPA), methyl isobutyl ketone, tetrahydrofuran, ethyl acetate, dichloromethane, dichloromethane:ethanol:isopropanol (5:6:4), and/or the like, can be incorporated during the formulation process. Particularly useful solvents include ethanol and IPA.

The films can be prepared in a laminated series. For example, a series of thin film layers can be consecutively laid down to make thicker films, or films with alternate layers with different functions. In one embodiment, films are multilayered laminates incorporating separate layers comprising antacids or mucoadhesives. The first bioactive layer can be overlaid with a second film layer containing antacid of sufficient quantity to buffer the stomach acid of mammals. Optionally, the bioactive layer can be sandwiched between two antacid layers to aid in passing through the stomach into the intestines without substantial degradation. Antacid for incorporation can include, e.g., alkaline acetate, citrate, succinate, tartrate, maleate, lactate, ammonium bicarbonate, phosphate, magnesium oxide, aluminum oxide, aluminum hydroxide with magnesium hydroxide, aluminum carbonate gel, calcium carbonate, sodium bicarbonate, hydrotalcite, sucralfate, bismuth subsalicylate, and/or the like.

Application of the wet blend can be at an initial thickness adequate to provide the desired final thickness, e.g., depending on the wet blend total solids and desired final residual moisture. For example, the wet blend can be applied to the drying surface to a depth ranging from less than about 5 microns to more than about a centimeter, from 50 microns to 5 millimeters, from 250 microns to 2,500 microns, or about 500 microns. The dried product thickness will typically range in thickness from less than about 5% of the starting wet blend thickness to more than 50% of the starting thickness, from 10% to 30%, or about 15% of the starting thickness.

Removal of the dried film from the drying surface can be facilitated by adjustments to the film formulation, choice of drying surface material, and/or utilization of a release coating on the drying surface. For example, the formulation can include a surfactant (e.g. Tween 80), the drying surface can be polyethylene terephthalate (PET) or a fluoropolymer; or the surface can be coated with a light lubricant, such as a silicone oil, plant oil, or mineral oil.

### Preferred Combinations of Constituents and Process Parameters

Certain combinations of formula constituents are more effective in stabilizing bioactive agents in processes and dried thin films of the invention. Typically, the dried films will include the bioactive agent, a sugar, a buffer, and a matrix polymer. For various bioactive agents, we have identified suitable formulations for production, storage, and administration. These formulations have certain common elements and certain alternate elements. Several formulation constituents, concentrations, and proportions have been found to have unexpected benefits in the context of dried bioactive films.

One of skill in the art understands that the materials, formulations, and methods described herein can be used in various functional combinations with an expectation of success, based on the teachings herein. For example, the disclosed bioactive agents can be combined with the disclosed excipient solutions and matrix polymers for drying of a thin film. Of course, some combinations will work better than others, but the majority will retain activity, and every embodiment has a different tradeoff between desirable but conflicting parameters. That is, most of the combinations of described elements are expected to function (without undue experimentation), but offer a different set of desirable characteristics (activity, pliability, dissolution rates, recovery, stability, etc.).

Quick-dissolving (i.e., dissolution within less than ˜2 minutes, under the tongue or in a standard dissolution apparatus, as known in the art) thin film compositions can be prepared by providing one or more bioactive agents (e.g. viral vaccine, a bacterial vaccine, a nucleic acid, a protein, an antibody, an enzyme, a growth factor, a cytokine, an adjuvant, or a virus-like particle); providing one or more pharmaceutically acceptable excipient solutions (e.g., any of the listed formulations F1 to F24, M1 to M7, T1 and T2); providing a matrix polymer stock (e.g., polyvinyl alcohols (PVA), alginate, polyethylene oxide, poly vinyl pyrrolidone, and/or poloxamer); combining the bioactive agent with the excipients in a solution or suspension; combining the solution or suspension with the one or more matrix polymers to form a wet blend; applying the wet blend to a flat surface; and, drying the wet blend to form a dry thin film. The drying step can go on, e.g., at a temperature from 20° C. to 60° C. for 1 to 4 hours (e.g., until the residual moisture is between 2 and 5%).

A number of methods and compositions are discussed in this Detailed Discussion and in the Examples section. As would be readily appreciated by the skilled person, the disclosures can be read in combination.

### EXAMPLES

The following examples are offered to illustrate, but not to limit the claimed invention. Immediately below is an index listing of the various examples in this section.

Example 1—Potency Testing of OTF's by Fluorescence Focus Assay (FFA).

Example 2—Excipient Solutions for Preparation of Bioactive Stock Solutions.

Example 3—Ambient Thin Film Drying at Various Polymer Ratios.

Example 4—Varying Matrix Polymer Mixes with Ambient Drying.

Example 5—Varying Matrix Polymer Mixes with Heat Drying.

Example 6—Varying Matrix Polymer Mixes with Vacuum Drying.

Example 7—Convective Drying in the Presence of Non-Aqueous Solvents.

Example 8—Vacuum Drying in the Presence of Non-Aqueous Solvents.

Example 9—Wet Blend Stability.

Example 10—Matrix Polymer to Excipient Ratios.

Example 11—Alternate Matrix Polymers.

Example 12—Mechanical Properties with Dryness Levels.

Example 13—Accelerated Stability at 45° C.

Example 14—Short Drying Times: Residual Moisture and Stability.

Example 15—The Impact of Longer Drying Times on Residual Moisture and Stability.

Example 16—Combining Convection Heat and Vacuum Drying.

Example 17—Testing Additional Rotavirus Strains.

Example 18—Encasement of multiple vaccine types on OTF.

Example 19—The Impact of Residual Moisture Content on the Molecular Mobility Within Films.

Example 20A: Excipient Screening of Films with Dispersed Solid Antacid. Example 20B: Films with Dispersed Solid Antacid.

Example 21—OTFs with Monoclonal Antibody Bioactive Agents.

Example 22—OTFs with Bacteria.

Example 23—OTFs with Influenza Virusl Bioactive Agent.

Example 24—Production of Spray Dried Powder Bioactive Agent.

Example 25—OTFs Using Organic Solvents and Spray Dried Bioactive Agent.

Example 26—Immunogenicity study of OTF formulation in mice.

### Example 1—Potency Testing of OTF's by Fluorescence Focus Assay (FFA)

A monolayer of confluent MA-104 cells (derived from rhesus monkey kidney tissue obtained from American Type Culture Collection, Manassas, Va.) were grown in 96-well plates for 3-4 days in a medium supplemented with 10% Fetal Bovine Serum (FBS) and kept in a humidified incubator at 37° C., 5% CO₂. The old media was replaced with fresh media before infection with the virus. The sterile Oral Thin Film virus sample was transferred into a 10 mL sterile serum glass vial where it was reconstituted with the assay media, MEM/EBBS (Minimum Essential Medium with Earle's Balanced Salt and supplemented with L-Glutamine and Non-Essential Amino Acid) to its target potency concentration by swirling until it was a homogeneous solution. An aliquot of the sample was then activated in 5 μg/mL trypsin diluted in assay media for one hour in a humidified incubator at 37° C., 5% CO₂, then serially diluted four-fold in the assay media. The virus sample was further diluted four-fold when plating onto the 96-well MA-104 assay plates leaving some wells as cell controls (without the virus).

The infected plates were incubated for 18 hours in a humidified incubator at 36° C., 5% CO₂ to allow replication of the virus. At post-incubation, the cell monolayer was washed with fresh media and then fixed with 80% acetone in −20° C. The plates were air-dried for one hour after fixing. The monoclonal primary antisera specific for the detection of the rotavirus strains were prepared in PBS with 1% BSA at pre-determined concentrations. Fifty microliters of the diluted antisera were added to each well of the assay plate and kept in a humidified incubator at 37° C. for one hour. The plates were washed with PBST (phosphate buffered saline with tween) after the incubation with primary antibody. Fifty microliters of Alexa Fluor® 488 labeled secondary antibody (Thermo Fisher Scientific) diluted in PBS with 1% BSA were added to each well of the plate and kept in a 37° C. incubator for one hour.

The plates were finally washed with PBST and kept protected from light. The fluorescing cells were counted using an inverted Leica microscope equipped with appropriate lamp at 10× magnification. Virus dilutions containing approximately 20 to 150 fluorescent foci per field were used for counting. The fluorescent forming unit (FFU/mL) was calculated based on the number of fluorescent cells, virus dilution, magnification, and the surface area of the field counted.

### Example 2—Excipient Solutions for Preparation of Bioactive Stock Solutions

In the next several examples OTF's were fabricated using alternative processing conditions, compositions of film formers, and excipient profiles to investigate the impact on the process loss and storage stability of biologic potency of live rotavirus vaccines. A list of the chemical components used in the various excipient profiles tested is provided in Table 1.

TABLE 1
Excipient profile of rotavirus stock solution formulations
prior to blending with polymer stock solution.

| Excipient | | Citric | | | | | | | PolySorbate |
|---|---|---|---|---|---|---|---|---|---|
| Profile | KPO₄ | Acid | Sucrose | Sorbitol | Glycerin | CaCl₂ | ZnCl₂ | Gelatin | 80 |
| Designation | (mM) | (wt %) | (wt %) | (wt %) | (wt %) | (mM) | (mM) | (wt %) | (wt %) |
| F1 | 50 | 0.8 | 20 | 1.6 | 0 | 4 | 4 | 4 | 0 |
| F2 | 50 | 0.8 | 20 | 0 | 0 | 4 | 4 | 4 | 0 |
| F3 | 50 | 0.8 | 20 | 5 | 0 | 4 | 4 | 4 | 0 |
| F4 | 50 | 0.8 | 5 | 0 | 0 | 4 | 4 | 4 | 0 |
| F5 | 50 | 0.8 | 20 | 5 | 0 | 4 | 4 | 0 | 0 |
| F6 | 50 | 0.8 | 20 | 5 | 0 | 0 | 0 | 4 | 0 |
| F7 | 50 | 0.8 | 5 | 5 | 0 | 4 | 4 | 4 | 0 |
| F8 | 50 | 0.8 | 20 | 13 | 0 | 4 | 4 | 4 | 0 |
| F9 | 50 | 0.8 | 20 | 0 | 6 | 4 | 4 | 4 | 0 |
| F10 | 50 | 0.8 | 20 | 0 | 6 | 4 | 4 | 0 | 0 |
| F11 | 50 | 0.8 | 5 | 0 | 6 | 4 | 4 | 4 | 0 |
| F12 | 50 | 0.8 | 20 | 0 | 4 | 4 | 4 | 4 | 0 |
| F13 | 50 | 0.8 | 20 | 0 | 12 | 4 | 4 | 4 | 0 |
| F14 | 50 | 0.8 | 10 | 0 | 6 | 4 | 4 | 0 | 0 |
| F15 | 50 | 0.8 | 5 | 0 | 6 | 4 | 4 | 0 | 0 |
| F16 | 50 | 0.8 | 5 | 5 | 0 | 4 | 4 | 0 | 0 |
| F17 | 50 | 0.8 | 5 | 10 | 0 | 4 | 4 | 0 | 0 |
| F18 | 50 | 0 | 7.5 | 0 | 0 | 0 | 0 | 0 | 0 |
| F19 | 50 | 0.8 | 20 | 0 | 0 | 4 | 4 | 2 | 0 |
| F20 | 50 | 0.8 | 6 | 0 | 0 | 4 | 4 | 2 | 0 |
| F21 | 50 | 0.8 | 20 | 0 | 25 | 4 | 4 | 4 | 0 |
| F22 | 50 | 0.8 | 30 | 0 | 25 | 4 | 4 | 4 | 0 |
| F23 | 50 | 0.8 | 10 | 0 | 6 | 4 | 4 | 4 | 0 |
| F24 | 50 | 0.8 | 20 | 5 | 0 | 4 | 4 | 4 | 0.1 |

In this example live rotavirus-containing OTF's in the presence of selected pharmaceutical excipients as stabilizers were evaluated for their ability to maintain potency through processing relative to a formulation with limited excipients (only sucrose and a buffer). The methods are described below:

Live monovalent rotavirus vaccine was aseptically formulated in limited pharmaceutical stabilizers: 7.5% sucrose and 50 mM potassium phosphate at pH 6.3 (formulation ‘F18’) to a titer of 6.5 log ffu/mL. A second preparation was aseptically formulated in a full complement of pharmaceutical stabilizers: 20% sucrose, 50 mM potassium phosphate at pH 6.3, 2% gelatin (GELITA®, VacciPro), 4 mM zinc chloride, 4 mM calcium chloride, and 0.8% citric acid (formulation ‘F19’) to a titer of 6.5 log ffu/mL. In another container, 12 parts of 4% solids content sodium alginate (Sigma-Aldrich, viscosity 15-20 cP at 1% in water), 1 part of 4% solids content sodium citrate, 4 parts of 1% solids content polyethylene oxide (TEO′, Sigma-Aldrich, Mv˜100,000), and 2 parts of 4% solids content polyvinyl alcohol (TVA′, Sigma-Aldrich, Mw˜146,000-186,000) was aseptically mixed to create a polymer mixture of film formers (‘P10’) with 3.37% solids content. Then, 8 parts of the formulated rotavirus solution (either F18 or F19) was added to 19 parts of the polymer mixture P10. This OTF ‘wet blend’ was dispensed into a circular dish and dried for 3 hours in a sterile tissue culture laminar flow hood at room temperature. The FFA assay (Example 1) was performed to determine the titer of the vaccine.

TABLE 2
Rotavirus process loss for varying excipient profiles
in dried films following ambient laminar flow drying

| | Rotavirus Process |
|---|---|
| Excipient Profile | Foss (log ffu) |
| F18 (sucrose + buffer) | >1.47 |
| F19 (sucrose + | 0.35 |
| buffer + stabilizers) | |

The results in Table 2 illustrate the benefit of the more complete excipient profile (F19) in protecting the virus through processing and reducing process loss.

### Example 3—Ambient Thin Film Drying at Various Polymer Ratios

Following a similar approach to Example 2, live rotavirus-containing OTF's in the presence of different total concentration of pharmaceutical stabilizers, but with the same relative amounts, were evaluated for their ability to maintain potency through processing. The preparation methods are described below:

Live monovalent rotavirus vaccine was aseptically formulated at a titer of 6.5 log ffu/mL in the following pharmaceutical stabilizers: 4 mM zinc chloride, 4 mM calcium chloride, 0.8% solids content citric acid, 2% solids content gelatin, 50 mM potassium phosphate pH of 6.3, and 6% solids content sucrose (Formulation ‘F20’). In another container, the polymer mixture P10 was prepared as described in Example 2. Then, 1 to 8 parts of the formulated rotavirus solution was added to 19 parts of the polymer mixture to provide the values indicated in Table 3. This OTF wet blend was dispensed into a circular dish and dried for 3 hours in a sterile tissue culture laminar flow hood at ambient conditions. The FFA assay (Example 1) was performed to determine the titer of the vaccine.

TABLE 3
Rotavirus process loss for varying ratios of film
formers to pharmaceutical stabilizers in dried
films following ambient laminar flow drying

| # Parts | Weight | Total Weight | |
|---|---|---|---|
| Formulated | Percent | Percent | |
| Vaccine to 19 | Polymeric | Pharmaceutical | Rotavirus |
| Parts | Solids | Stabilizers | Process |
| Polymer | in Casting | in the | Loss |
| Mixture | Solution | Dried Film | (log ffu) |
| 1 | 3.20% | 13.5% | Sample Below |
| | | | Detection Limit |
| | | | (BDL) |
| 2 | 3.05% | 23.8% | >1.2 |
| 4 | 2.78% | 38.4% | 1.08 |
| 8 | 2.37% | 53.13% | 0.35 |

The results demonstrate the stabilizing effect of the higher loading of excipients in the final film, at the expense of the content of polymeric film-formers, to reduce process loss.

### Example 4—Varying Matrix Polymer Mixes with Ambient Drying

In this example live rotavirus-containing OTF's in the presence of a fixed profile of pharmaceutical stabilizers, but with varying polymer compositions, were evaluated for their ability to maintain potency through processing. The preparation methods are described below:

Live monovalent rotavirus vaccine was aseptically formulated at a titer of 6.4 log ffu/mL in formulation F20. Sodium alginate, sodium citrate, polyethylene oxide (PEO), and polyvinyl alcohol (PVA) was aseptically mixed as indicated in Table 4. Then the formulated rotavirus solution was added to the polymer mixture to achieve a titer of 5.87 log ffu/mL. This OTF wet blend was dispensed into a circular dish and dry for 18 hours in a laminar flow hood at ambient conditions. The solids content shown in Table 4 represents the final weight percentages in the dry film; these values plus the solids from the formulated vaccine constitute 100% of solids in dry film. The FFA assay (Example 1) was performed to determine the titer of the vaccine.

TABLE 4
Rotavirus process loss for varying polymer component concentrations
in dried film following ambient laminar flow drying

| | | | | | Rotavirus |
|---|---|---|---|---|---|
| Polymer | Sodium | Sodium | | | Process |
| Composition | alginate | citrate | PEO | PVA | Loss |
| # | (wt %) | (wt %) | (wt %) | (wt %) | (log ffu) |
| P10 | 35.15% | 2.93% | 2.93% | 5.86% | 0.46 ± 0.171 |
| P20 | 39.29% | 2.18% | 2.18% | 4.37% | 0.90 |
| P23 | 37.71% | 3.14% | 3.14% | 6.28% | 0.60 ± 0.364 |
| P24 | 37.49% | 3.12% | 3.12% | 3.12% | 0.71 ± 0.345 |

Although there is some variability in the process loss for the different polymer formulations, the results do not show a strong effects over the ranges evaluated.

### Example 5—Varying Matrix Polymer Mixes with Heat Drying

Similar to Example 4 but utilizing convection heat drying of the films, live rotavirus-containing OTF's in the presence of a fixed profile of pharmaceutical stabilizers with varying polymer compositions were evaluated for their ability to maintain potency through processing. The preparation methods are described below:

Live monovalent rotavirus vaccine was aseptically formulated at a titer of 6.4 log ffu/mL in formulation F20. Sodium alginate, sodium citrate, polyethylene oxide (PEO), polyvinyl alcohol (PVA), and polyvinyl pyrrolidone (PVP, Kollidon 90 F, Mv˜1,100,000) was aseptically mixed as indicated in Table 5. The formulated rotavirus solution was added to the polymer mixture to achieve a titer of 5.87 log ffu/mL. The resulting OTF wet blend was dispended into a circular dish and dried for 3 hours by 50° C. convective flow with a Duracraft ceramic heater. The solids content shown in Table 5 represents the final weight percentages in the dry film; these values plus the solids from the formulated vaccine constitute 100% of solids in dry film. The FFA assay (Example 1) was performed to determine the titer of the vaccine.

TABLE 5
Rotavirus process loss for varying polymer component concentrations
in dried film following heated convection drying at 50° C.

| Polymer | Sodium | Sodium | | | | Rotavirus |
|---|---|---|---|---|---|---|
| Composition | Alginate | Citrate | PEO | PVA | PVP | Process Loss |
| # | (wt %) | (wt %) | (wt %) | (wt %) | (wt %) | (log ffu) |
| P10 | 35.15% | 2.93% | 2.93% | 5.86% | 0% | 0.41 ± 0.067 |
| P20 | 39.29% | 2.18% | 2.18% | 4.37% | 0% | >1.83 |
| P23 | 37.71% | 3.14% | 3.14% | 6.28% | 0% | 0.92 ± 0.403 |
| P24 | 37.49% | 3.12% | 3.12% | 3.12% | 0% | 0.64 ± 0.206 |
| P29 | 0% | 0% | 14.68% | 0% | 58.70% | Not determined |
| P30 | 42.95% | 2.37% | 2.39% | 0% | 0% | 0.18 ± 0.081 |
| P31 | 43.91% | 2.44% | 0% | 4.88% | 0% | No loss |

The results demonstrate that for certain polymer compositions (i.e. P30 and P31) the films can be produced with higher temperature drying over a shorter amount of time while maintaining potency.

### Example 6—Varying Matrix Polymer Mixes with Vacuum Drying

Here, Example 5 was repeated replacing the heated convection drying with vacuum drying. The preparation methods are described below:

Live monovalent rotavirus vaccine was aseptically formulated with pharmaceutical stabilizers and combine with polymer components as described in Example 5. This film wet blend was dispensed into a circular dish and dried under vacuum while maintaining the sample temperature at 25° C. for 1 hour (Vacuum at 100 Torr for 20 min, then 50 Torr for 20 min, and then 20 min at 25 Torr). Then the temperature was increased one degree per minute for 12 minutes to 37° C. Temperature was kept at 37° C. for 2 hours. The solids content shown in Table 6 represents the final weight percentages in the dry film; these values plus the solids from the formulated vaccine constitute 100% of solids in dry film. The FFA assay (Example 1) was performed to determine the titer of the vaccine.

TABLE 6
Rotavirus process loss for varying polymer component
concentrations in dried film following vacuum drying

| Polymer | Sodium | Sodium | | | | Rotavirus |
|---|---|---|---|---|---|---|
| Composition | Alginate | Citrate | PEO | PVA | PVP | Process Loss |
| # | (wt %) | (wt %) | (wt %) | (wt %) | (wt %) | (log ffu) |
| P29 | 0% | 0% | 14.68% | 0% | 58.70% | 0.3 |
| P30 | 42.95% | 2.37% | 2.39% | 0% | 0% | 0.34 ± .080 |
| P31 | 43.91% | 2.44% | 0% | 4.88% | 0% | 0.06 ± 0.194 |

Relative to the results of the previous example, the use of vacuum drying did not significantly alter the process loss, therefore providing a viable alternative for rapid drying without higher temperatures.

### Example 7—Convective Drying in the Presence of Non-Aqueous Solvents

In this example two non-aqueous solvents (ethanol and isopropanol) were added to the film casting solution prior to casting and drying to enhance drying kinetics. Otherwise, the preparation methods were similar to Example 5.

Live monovalent rotavirus vaccine was aseptically formulated in pharmaceutical stabilizers as described in Example 4. Sodium alginate, sodium citrate, polyethylene oxide (PEO), and polyvinyl alcohol (PVA) was aseptically mixed as indicated in Table 7. To the polymer mixture, the solvent indicated in Table 7 was added so that solvent was fifteen percent of the final volume (including the rotavirus mixture). The formulated rotavirus mixture was added to the polymer mixture to achieve a rotavirus titer of 5.87 log ffu/mL. This film wet blend was dispensed into a circular dish and dried for 3 hours with convective flow at 50° C. using a Duracraft ceramic heat furnace. The solids content shown in Table 7 represents the final weight percentages in the dry film; these values plus the solids from the formulated vaccine constitute 100% of solids in dry film. The FFA assay (Example 1) was performed to determine the titer of the vaccine.

TABLE 7
Rotavirus process loss in dried film following solvent-enhanced drying

| Polymer | Sodium | Sodium | | | | Rotavirus |
|---|---|---|---|---|---|---|
| Composition | Alginate | Citrate | PEO | PVA | | Process Loss |
| # | (wt %) | (wt %) | (wt %) | (wt %) | Solvent | (log ffu) |
| P20 | 39.29% | 2.18% | 2.18% | 4.37% | none | >1.83 |
| P20 | 39.29% | 2.18% | 2.18% | 4.37% | Water | 0.93 ± 0.160 |
| P20 | 39.29% | 2.18% | 2.18% | 4.37% | Ethanol | 0.24 ± 0.247 |
| P20 | 39.29% | 2.18% | 2.18% | 4.37% | Isopropanol | 0.38 |

The results indicate that the addition of the non-aqueous, more volatile solvents such as ethanol or isopropanol can potentially reduce process losses, possibly by improving the drying kinetics.

### Example 8—Vacuum Drying in the Presence of Non-Aqueous Solvents

Here Example 7 was repeated replacing the heated convection drying with vacuum drying. The methods are otherwise similar:

The live monovalent rotavirus vaccine was aseptically formulated in pharmaceutical stabilizers and combined with the polymer mixture and solvent as described in Example 7. This film wet blend was dispensed into a circular dish and dried under vacuum while maintaining the sample temperature at 25° C. for 2.5 hours (Vacuum at 100 Torr for 45 min, then 50 Torr for 45 min, and then 1 hour at 25 Torr). The solids content shown in Table 8 represents the final weight percentages in the dry film; these values plus the solids from the formulated vaccine constitute 100% of solids in dry film. The FFA assay (Example 1) was performed to determine the titer of the vaccine.

TABLE 8
Rotavirus process loss in dried film following solvent-enhanced vacuum drying

| Polymer | Sodium | Sodium | | | | Rotavirus |
|---|---|---|---|---|---|---|
| Composition | Alginate | Citrate | PEO | PVA | | Process Loss |
| # | (wt %) | (wt %) | (wt %) | (wt %) | Solvent | (log ffu) |
| P20 | 39.29% | 2.18% | 2.18% | 4.37% | Water | No loss |
| P20 | 39.29% | 2.18% | 2.18% | 4.37% | Ethanol | No loss |
| P20 | 39.29% | 2.18% | 2.18% | 4.37% | Isopropanol | No loss |

These data demonstrate vacuum drying conditions that result in minimal process loss, regardless of the solvents used.

### Example 9—Wet Blend Stability

In this example live monovalent rotavirus vaccine G3 strain was formulated with a number of pharmaceutical stabilizers and film-forming polymer into an aqueous wet blend. The short-term wet blend stability was evaluated at various temperatures. The wet blend was also fabricated into thin films using different drying temperatures and evaluated for their process loss in titer. The methods to produce and test the films are described as follows:

Live monovalent rotavirus vaccine was aseptically formulated to a titer of 7.0 log ffu/mL with an aqueous excipient stock solution of pharmaceutical stabilizers, pH-adjusted to 6.2-6.5 with 1N KOH, such that the resulting viral stock solution composition (T9′) was: 4 mM calcium chloride, 4 mM zinc chloride, 0.8% citric acid, 4% gelatin (GELITA® VacciPro), 20% sucrose, 6% glycerin, and 50 mM potassium phosphate. In a 1:1 ratio this rotavirus stock solution was mixed with a polymer mixture T1′ composed of a 25% by weight aqueous solution of polyvinyl alcohol (Sigma-Aldrich, Mw-67,000). The film wet blends were degassed by centrifugation at a speed of 1000 rcf (relative centrifugal force) for 2 minutes.

Portions of the resulting rotavirus film wet blend were dispensed into 4 separate vials. Each vial was placed into different water baths each at a different temperature: 4, 40, 45 and 50° C. for up to one hour. The vials were removed from the water baths and the titer of the stored wet blends was measured by the FFA assay described in Example 1. The assay results provided in Table 9A indicate the wet blend is very unstable at 50° C., losing almost 1 log in titer in just 15 minutes relative to the same wet blend stored at lower temperatures (4 to 45° C.) for one hour.

TABLE 9A
Measured film wet blend titer after storage
in water baths at different temperatures

| Water Bath | Time of Storage | Measured Titer |
|---|---|---|
| Temperature | in Water Bath | after Storage |
| (° C.) | (Minutes) | (log ffu/ml) |
| 4 | 60 | 6.52 |
| 40 | 60 | 6.72 |
| 45 | 60 | 6.61 |
| 50 | 15 | 5.60 |

The remaining wet blend was cast as three separate films on polyethylene terephthalate (PET) backing liners (Kinmar PET, K-Mac Plastic) using a manual applicator (BYK-Gardner) for a depth of 20 mil. The wet films were dried for 0.5 to 4 hours at 50, 60, or 70° C. in a convection oven (VWR, model 1350FM).

Films delaminated easily from the liner and were flexible and smooth without depressions. The dried films were sectioned into approximately 100 mg portions. Rotavirus titer was determined as described in Example 1. The film thickness (measured by a QUALITEST thickness gauge, model FM 101, average of three measurements), the moisture content (measured by Karl Fischer titration using a Aquacounter AQ-200 Coulometric Titrator with Hydranal Coulomat AG and Hydranal Coulomat CG as anode and cathode solutions, respectively, and with the former also used as the film extraction solvent) and the rotavirus titer loss observed for the process from the wet film to the dried film are provided in Table 9.

TABLE 9
Physical properties and process loss for an OTF formulation
containing rotavirus vaccine dried at different temperatures

| | | | | | | |
|---|---|---|---|---|---|---|
| Drying time | 0.5 | 0.5 | 0.5 | 1.0 | 2.0 | 4.0 |
| (hours) | | | | | | |
| Drying | 50 | 60 | 70 | 50 | 50 | 50 |
| Temperature | | | | | | |
| (° C.) | | | | | | |
| Film thickness | 77 | 66 | 101 | 80 | 70 | 73 |
| (μm) | | | | | | |
| Moisture content | 8.3 | Not | Not | 6.0 | 4.7 | 2.5 |
| (wt %) | | measured | measured | | | |
| Process Loss | 0 | 0 | 0.5 | 0 | 0 | 0.1 |
| (log ffu) | | | | | | |

These results indicate that although the formulated aqueous liquid film wet blend is very unstable at 50° C. for as little as 15 minutes, drying the film at this temperature, or even higher at 60° C., for as much as 4 hours does not cause any significant loss in titer due to process loss. The drying process allows for evaporative cooling to protect the virus at these temperatures until the moisture content is low enough to significantly reduce the molecular mobility of the film and provide further protection to the virus. These results further demonstrate the superior thermal stability of the film formulation relative to a liquid formulation, despite both containing the same excipient stabilizers. However, rotavirus vaccine is partially inactivated in a half hour by film drying at 70° C., which appears to be approximately the upper limit in temperature for this drying process in terms of low process loss in viral potency.

### Example 10—Matrix Polymer to Excipient Ratios

Live monovalent rotavirus vaccine, which contained the G3 strain, was incorporated into OTF's with different concentrations of pharmaceutical excipients to evaluate process loss for a wider range of polymer to excipient ratio. The procedures for preparation were as follows:

Live monovalent rotavirus vaccine (G3 strain) was aseptically formulated to a titer of 7.0 log ffu/mL with formulations F9, F21 (4 mM calcium chloride, 4 mM zinc chloride, 0.8% citric acid, 4% gelatin (GELITA® VacciPro), 20% sucrose, 25% glycerin, and 50 mM potassium phosphate), F22 (4 mM calcium chloride, 4 mM zinc chloride, 0.8% citric acid, 4% gelatin, 30% sucrose, 25% glycerin, and 50 mM potassium phosphate), and F23 (4 mM calcium chloride, 4 mM zinc chloride, 0.8% citric acid, 4% gelatin, 10% sucrose, 6% glycerin, and 50 mM potassium phosphate) (see Table 1), preparing the film wet blends as in Example 9.

The rotavirus film wet blends were cast on a PET backing liner as in Example 9. The wet films were dried for 30 minutes at 50° C. in a convection oven (VWR, model 1350FM). The titers were determined by the FFA assay method described in Example 1. The films produced were flexible and smooth without depressions. The film thickness and titer loss observed for the process from the wet film to the dried film are provided in Table 10.

TABLE 10
Physical properties and process loss for OTF formulation containing
rotavirus vaccine with different excipient loadings

| Excipient Profile | F22 | F21 | F9 | F23 |
|---|---|---|---|---|
| Polymeric (PVA) | 29.2 | 33.1 | 44.2 | 53.7 |
| loading of | | | | |
| dried film | | | | |
| (wt %) | | | | |
| Glycerin loading | 29.2 | 33.1 | 10.6 | 12.9 |
| of dried film | | | | |
| (wt %) | | | | |
| Film | 87 | 85 | 85 | 77 |
| thickness (μm) | | | | |
| Process Loss | >2.2 | >2.3 | 0.0 | 0.25 |
| (log ffu) | | | | |

These results indicate that the rotavirus vaccine is inactivated by film processing/drying in the presence of high solids content of plasticizing stabilizers, particularly glycerin, at the expense of the polymeric film former.

### Example 11—Alternate Matrix Polymers

In this example OTF formulations containing a number of pharmaceutical stabilizers and alternative film-forming polymers were fabricated to evaluate their suitability in terms of mechanical properties. The methods to produce them are described as follows:

Aqueous excipient stock solutions of pharmaceutical stabilizers were aseptically formulated and pH-adjusted to 6.2-6.5 with 1N KOH, such that the resulting composition was either F3 (4 mM calcium chloride, 4 mM zinc chloride, 0.8% citric acid, 4% gelatin (GELITA® VacciPro), 5% sorbitol, 20% sucrose, and 50 mM potassium phosphate) or F24 (4 mM calcium chloride, 4 mM zinc chloride, 0.8% citric acid, 4% gelatin, 5% sorbitol, 20% sucrose, 0.1% Tween 80 and 50 mM potassium phosphate) (see Table 1). In a 1:1 ratio the F3 stock solution was aseptically mixed with a polymer mixture T2′ composed of 24% by weight aqueous solution of hydroxypropyl methylcellulose (HPMC, hydroxylpropoxyl content ˜9%; Sigma-Aldrich product No. 09963); the F24 stock solution was similarly mixed with a polymer mixture T3′ composed of 30% polyvinyl pyrrolidone (PVP, Kollidon® 90F, BASF). The two resulting film wet blends were degassed by centrifugation at a speed of 1000 rcf (relative centrifugal force) for 2 minutes.

The film wet blends were cast as two separate films on polyethylene terephthalate (PET) backing liners (Kinmar PET, K-Mac Plastic) using a manual applicator (BYK-Gardner) for a depth of 20 mil. The wet films were dried for 60 minutes at 60° C. in a convection oven (VWR, model 1350FM).

The film made from HPMC (P2) was notably phase separated and cloudy. The film made from PVP (P3) was brittle and difficult to delaminate from the liner.

### Example 12—Mechanical Properties with Dryness Levels

In this example an OTF formulation containing a number of pharmaceutical stabilizers (F3) and a film-forming polymer (P1) was fabricated with different drying conditions to evaluate drying kinetics and their suitability in terms of mechanical properties. The methods to produce them are described as follows:

Here F3 aqueous excipient stock solution of pharmaceutical stabilizers was aseptically formulated and pH-adjusted to 6.2-6.5 with 1N KOH (see Table 1). In a 1:1 ratio the F3 stock solution was aseptically mixed with polymer mixture T1′ to prepare the film wet blend as described in Example 9.

The film wet blend was cast into several separate films on polyethylene terephthalate (PET) backing liners (Kinmar PET, K-Mac Plastic) using a manual applicator (BYK-Gardner) for a depth of 20 mil. The wet films were dried as described in Table 11 at 60° C. in a convection oven (VWR, model 1350FM). Some of the films (as indicated in Table 11) were exposed to additional vacuum drying at 100 mTorr and 4° C. The mechanical properties of the resulting dried films are described in Table 11.

TABLE 11
Convection oven drying kinetics and mechanical properties of
OTF from the F3 formulation with P1 polymer

| | Vacuum | | |
|---|---|---|---|
| Drying | time @ | | |
| Time @ | 100 mTorr, | Moisture | |
| 60° C. | 4° C. | Content | Mechanical |
| (hr) | (hr) | (wt %) | Properties |
| 1 | 0 | 3.84 | Flexible |
| 2 | 0 | 2.58 | Brittle |
| 3 | 0 | 2.25 | Brittle |
| 2 | 24 | 2.02 | Very brittle, difficult |
| | | | to delaminate |
| 2 | 48 | 1.77 | Very brittle, very |
| | | | difficult to |
| | | | delaminate |

The findings indicate that, for this formulation, drying at 60° C. for 2 hours or more results in moisture content less than 3% and in brittle films that are difficult to delaminate.

### Example 13—Accelerated Stability at 45° C.

Live monovalent rotavirus vaccine, which contained the G3 strain, was incorporated into OTF's with different concentrations of pharmaceutical excipients to evaluate process loss and storage stability at 45° C. The procedures for preparation were as follows:

Live monovalent rotavirus vaccine (G3 strain) was aseptically formulated to a titer of 7.0 log ffu/mL with formulations F1 (4 mM calcium chloride, 4 mM zinc chloride, 0.8% citric acid, 4% gelatin (GELITA® VacciPro), 20% sucrose, 1.6% sorbitol, and 50 mM potassium phosphate), F2 (4 mM calcium chloride, 4 mM zinc chloride, 0.8% citric acid, 4% gelatin, 20% sucrose, and 50 mM potassium phosphate), F3 (4 mM calcium chloride, 4 mM zinc chloride, 0.8% citric acid, 4% gelatin, 5% sorbitol, 20% sucrose, and 50 mM potassium phosphate), F4 (4 mM calcium chloride, 4 mM zinc chloride, 0.8% citric acid, 4% gelatin, 5% sucrose, and 50 mM potassium phosphate), F5 (4 mM calcium chloride, 4 mM zinc chloride, 0.8% citric acid, 20% sucrose, 5% sorbitol, and 50 mM potassium phosphate), F6 (0.8% citric acid, 4% gelatin, 5% sorbitol, 20% sucrose, and 50 mM potassium phosphate), F7 (4 mM zinc chloride, 4 mM calcium chloride, 0.8% citric acid, 4% gelatin, 5% sorbitol, 5% sucrose, and 50 mM potassium phosphate), and F8 (4 mM calcium chloride, 4 mM zinc chloride, 0.8% citric acid, 4% gelatin, 20% sucrose, 13% sorbitol, and 50 mM potassium phosphate) (see Table 1), preparing the film wet blends as described in Example 9.

The rotavirus film wet blends were cast on a PET backing liner as described in Example 9 at a depth of 20 mil for F1, F3, F6 and F8, 25 mil for F2 and F5, and 30 mil for F4 and F7. The wet films were dried for 1 hour at 60° C. in a convection oven (VWR, model 1350FM).

The dried films were sectioned into approximately 100 mg portions for an accelerated stability study at 45° C. for 8 to 20 weeks. The titers were determined by the FFA assay method described in Example 1. The films produced were flexible and smooth without depressions. The moisture content (measured by Karl Fischer titration), film thickness, titer loss observed for the process from the wet film to the dried film, and the rotavirus stability are provided in Table 12. Storage stability was measured by the slope of the best line from a plot of log ffu versus time.

TABLE 12
Physical properties and stability of OTF formulations containing G3
rotavirus vaccine after 1 hour drying at 60° C. in a convection oven

| | | | | Storage |
|---|---|---|---|---|
| | Film | | | Stability |
| | Moisture | Film | Process | @45° C. |
| Excipient | Content | thickness | Loss | (log ffu |
| Profile | % | (μm) | (log ffu) | loss/wk) |
| F1 | 5.0 | 77 | 0.0 | 0.06 |
| F2 | 5.9 | 86 | 0.0 | 0.06 |
| F3 | 5.2 | 79 | 0.0 | 0.07 |
| F4 | 6.3 | 79 | 0.0 | 0.04 |
| F5 | 5.5 | 100 | 0.0 | 0.16 |
| F6 | 5.4 | 85 | 0.0 | 0.04 |
| F7 | 6.4 | 82 | 0.0 | 0.06 |
| F8 | 4.4 | 83 | 0.0 | 0.10 |

While the film fabrication had negligible process loss for all formulations, the storage stability results were less clear, indicating possibly the negative impacts of gelatin removal from the formulation (F5) or high sorbitol content (F8).

### Example 14—Short Drying Times: Residual Moisture and Stability

In this example OTF's were fabricated using shorter drying times (30 minutes or less in a convection oven) to explore possible production methods more favorable for commercial manufacturing. A variety of live rotavirus G3 strain vaccine formulations were evaluated for their physical appearance and flexibility. Moisture content, process loss and storage stability were also recorded for some formulations. The methods are described below:

Live monovalent rotavirus vaccine (G3 strain) was aseptically formulated to a titer of 7.0 log ffu/mL with formulations F3, F9, F10 (4 mM calcium chloride, 4 mM zinc chloride, 0.8% citric acid, 20% sucrose, 6% glycerin, and 50 mM potassium phosphate), F11 (4 mM calcium chloride, 4 mM zinc chloride, 0.8% citric acid, 4% gelatin, 5% sucrose, 6% glycerin, and 50 mM potassium phosphate), F12 (4 mM calcium chloride, 4 mM zinc chloride, 0.8% citric acid, 4% gelatin, 20% sucrose, 4% glycerin, and 50 mM potassium phosphate), F13 (4 mM calcium chloride, 4 mM zinc chloride, 0.8% citric acid, 4% gelatin, 20% sucrose, 12% glycerin, and 50 mM potassium phosphate), F14 (4 mM calcium chloride, 4 mM zinc chloride, 0.8% citric acid, 10% sucrose, 6% glycerin, and 50 mM potassium phosphate), and F15 (4 mM calcium chloride, 4 mM zinc chloride, 0.8% citric acid, 5% sucrose, 6% glycerin, and 50 mM potassium phosphate) (see Table 1), preparing the film wet blends as in Example 9.

The rotavirus film wet blends were cast on a PET backing liner as in Example 9. The wet films were dried for 15 or 30 minutes at 50 or 60° C. in a convection oven (VWR, model 1350FM).

The dried films were sectioned into approximately 100 mg portions, with some participating in a 4-5 week accelerated stability study at 45° C. The titers were determined by the FFA assay method described in Example 1. The films produced were flexible and smooth without depressions. The moisture content (measured by Karl Fischer titration), film thickness, titer loss observed for the process from the wet film to the dried film, and the rotavirus stability are provided in Table 13.

TABLE 13
Physical properties and stability of OTF formulations containing G3
rotavirus vaccine for short-duration drying in a convection oven

| | | | | | | Storage |
|---|---|---|---|---|---|---|
| | Film | | | Film | | Stability |
| | Drying | Film | Film | Moisture | Process | @45° C. |
| Film | Time | Drying | thickness | Content | Loss | (log ffu |
| Formulation | (min.) | (° C.) | (μm) | (wt %) | (log ffu) | loss/wk) |
| F3 | 15 | 60 | 86 | 11.0 | 0.0 | 0.95 |
| F3 | 30 | 60 | 80 | 7.3 | 0.0 | 0.25 |
| F3 | 30 | 50 | 81 | Not | 0.0 | Not |
| | | | | measured | | measured |
| F9 | 30 | 60 | 66 | Not | 0.0 | Not |
| | | | | measured | | measured |
| F9 | 30 | 50 | 77 | 8.3 | 0.0 | 0.55 |
| F10 | 30 | 50 | 76 | 9.7 | 0.0 | 0.78 |
| F11 | 30 | 50 | 74 | 8.6 | 0.2 | 0.49 |
| F12 | 30 | 50 | 77 | Not | 0.0 | Not |
| | | | | measured | | measured |
| F13 | 30 | 50 | 99 | Not | 0.0 | Not |
| | | | | measured | | measured |
| F14 | 30 | 50 | 56 | Not | 0.0 | Not |
| | | | | measured | | measured |
| F15 | 30 | 50 | 57 | Not | 0.0 | Not |
| | | | | measured | | measured |

Again, the process loss for all the fabricated films was minimal for these drying conditions. However, the storage stability for the few that were measured indicated a reduction in stability, likely associated with increasing moisture content and/or absence of gelatin. In particular, for formulation F3, there was significantly reduced storage stability when moisture content increased from 7.3 to 11% owing to the shorter drying time.

### Example 15—The Impact of Longer Drying Times on Residual Moisture and Stability

In this example OTF's were fabricated using a longer drying time (2 hours in a convection oven) to explore production methods with lower drying temperature and/or providing reduced moisture content. Several film formulations containing live rotavirus G3 strain vaccine were evaluated for their physical appearance and flexibility. Moisture content, process loss and storage stability were also recorded for some formulations. The methods are described below:

Live monovalent rotavirus vaccine (G3 strain) was aseptically formulated to a titer of 7.0 log ffu/mL with formulations F3, F5, F16 (4 mM calcium chloride, 4 mM zinc chloride, 0.8% citric acid, 5% sucrose, 5% sorbitol, and 50 mM potassium phosphate), and F17 (4 mM calcium chloride, 4 mM zinc chloride, 0.8% citric acid, 5% sucrose, 10% sorbitol, and 50 mM potassium phosphate), preparing the film wet blends as in Example 9.

Rotavirus film wet blends were cast on a PET backing liners as in Example 9. Individual wet films were dried for 120 minutes at 50 or 60° C. in a convection oven (VWR, model 1350FM).

The dried films were sectioned into approximately 100 mg portions, with the F3 formulation participating in a 4-week accelerated stability study at 45° C. The titers were determined by the FFA assay method described in Example 1. The films produced were flexible and smooth without depressions, with the exception of the film produced at 60° C. which had some brittleness. The moisture content (measured by Karl Fischer titration), film thickness, titer loss observed for the process from the wet film to the dried film, and the rotavirus stability are provided in Table 14.

TABLE 14
Physical properties and stability of OTF formulations containing G3
rotavirus vaccine for long-duration drying in a convection oven.

| Excipient | | | | | |
|---|---|---|---|---|---|
| Profile | F3 | F5 | F9 | F16 | F17 |
| Drying | 60 | 50 | 50 | 50 | 50 |
| Temperature | | | | | |
| (° C.) | | | | | |
| Drying Time | 2 | 2 | 2 | 2 | 2 |
| (hour) | | | | | |
| Film Moisture | 2.9 | 5.8 | 4.7 | 6.4 | 6.1 |
| Content % | | | | | |
| Film Thickness | 107 | 74 | 70 | 101 | 90 |
| (μm) | | | | | |
| Process Loss | 0.0 | 0.0 | 0.0 | 0.2 | 0.0 |
| (log ffu) | | | | | |
| Storage | 0.04 | Not | Not | Not | Not |
| stabil- | | measured | measured | measured | measured |
| ity@45° C. | | | | | |
| (log ffu | | | | | |
| loss/wk) | | | | | |

The process loss for all these films was minimal for this longer drying time. However, the storage stability for the F3 formulation was not improved significantly by the resulting lower moisture content of 2.9%. The longer drying of 2 hours seemed to balance the lower convection oven temperature of 50° C. in terms of moisture content for the remaining formulations, versus 1 hour drying at 60° C.

### Example 16—Combining Convection Heat and Vacuum Drying

In this example OTF's were fabricated using the longer drying time (2 hours in a convection oven) in addition to drying under vacuum to investigate benefits of further reduced moisture content with preservation of viral potency. A number of film formulations containing live rotavirus G3 strain vaccine were evaluated for their physical appearance and flexibility. Moisture content, process loss and storage stability were also recorded. The methods are described below:

Live monovalent rotavirus vaccine (G3 strain) was aseptically formulated to a titer of 7.0 logs ffu/mL with formulations F1, F2, F3, and F8, preparing the film wet blend as in Example 9.

The rotavirus film wet blends were cast on a PET backing liner as in Example 9. The wet films were dried for 2 hours at 60° C. in a convection oven (VWR, model 1350FM), followed by an additional 24 hours drying at 100 mTorr vacuum at 4° C.

The dried films were sectioned into approximately 100 mg portions for an accelerated stability study at 45° C. for 8 to 14 weeks. The virus titers were determined by the FFA assay method described in Example 1. The films produced were smooth without depressions, but had some brittleness. The moisture content (measured by Karl Fischer titration), titer loss observed for the process from the wet film to the dried film, and the rotavirus stability are provided in Table 15.

TABLE 15
Physical properties and stability of OTF formulations
containing G3 rotavirus vaccine for long-duration
drying in a convection oven and exposure to vacuum.

| Excipient | | | | |
|---|---|---|---|---|
| Profile | F1 | F2 | F3 | F8 |
| Film Moisture | 2.6 | 2.7 | 3.1 | 2.6 |
| Content % | | | | |
| Film Thickness | Not | 89 | 86 | 75 |
| (μm) | measured | | | |
| Process Loss | 0.0 | 0.0 | 0.1 | 0.0 |
| (log ffu) | | | | |
| Storage | 0.02 | 0.04 | 0.07 | 0.08 |
| stability@45° C. | | | | |
| (log ffu loss/wk) | | | | |

The enhanced drying conditions produced films with lower moisture content without significant process loss. Most formulations showed benefit in storage stability as well. For formulation F3, comparing to the previous example, the addition of this vacuum drying step to the processing did not appear to provide a measurable reduction in moisture content. Since all of these formulations have the same excipient profile except for varying amounts of sorbitol, it does indicate that F1 may have the optimum amount of this excipient at an intermediate level.

### Example 17—Testing Additional Rotavirus Strains

OTF's were fabricated with two additional strains of the rotavirus vaccine, G1 and G2, to test the suitability of a given formulation across more than one strain. The F3 excipient profile was applied to each of these two strains and the resulting films were evaluated for their physical appearance and flexibility. Moisture content, process loss and storage stability were also recorded. The methods are described below:

Live monovalent rotavirus vaccine separately for G1 strain and for G2 strain were aseptically formulated to a titer of 7.0 log ffu/mL with formulation F3 preparing the film wet blends as in Example 9.

The rotavirus film wet blends were cast on a PET backing liner as in Example 9. The wet films were dried for 1 hour at 60° C. in a convection oven (VWR, model 1350FM).

The dried films were sectioned into approximately 100 mg portions for an accelerated stability study at 45° C. for 15 weeks. The titers were determined by the FFA assay method described in Example 1. The films produced were flexible and smooth without depressions. The moisture content (measured by Karl Fischer titration), titer loss observed for the process from the wet film to the dried film, and the rotavirus stability are provided in Table 16.

TABLE 16
Physical properties and stability of an OTF formulation
containing G1 and G2 strains of rotavirus vaccine
following drying in a convection oven

| | Film | Film | Process | Storage |
|---|---|---|---|---|
| Vaccine | Moisture | thickness | Loss | stability@45° C. |
| Strain | Content % | (μm) | (log ffu) | (log ffu loss/wk) |
| G1 | 4.6 | 74 | 0.1 | 0.05 |
| G2 | 4.7 | 75 | 0.0 | 0.01 |

The results indicate that the low process loss and storage stability provided by formulation F3 to the G3 strain (see Example 13) were similarly provided to two other strains of rotavirus: G1 and G2.

### Example 18—Encasement of multiple vaccine types on OTF

Two OTF batches were fabricated with a quadrivalent rotavirus vaccine containing the G1, G2, G3 and G4 strains to demonstrate the suitability of a given formulation for a multi-valent vaccine. The F2 and F3 excipient profiles were applied and the resulting films were evaluated for physical appearance and flexibility. Moisture content, process loss and storage stability were also recorded. The methods are described below:

Live quadrivalent rotavirus vaccine containing the G1, G2, G3, and G4 strains was aseptically formulated to a titer of 6.6 log ffu/mL/strain with F2 and separately with F3 formulation compositions (see Table 1) as described in Example 9. Also the individual film wet blends were prepared as described in Example 9.

The rotavirus wet film formulations were cast as in Example 9, but at a depth of 25 mil. The wet films were dried for 1 hour for F2 and 2 hours for F3 at 60° C. in a convection oven (VWR, model 1350FM).

The dried films were sectioned into approximately 100 mg portions for a 24-month storage stability study at 4° C., 25° C., and 40° C. The rotavirus titer was determined as described in Example 1. Films were flexible and smooth without depressions. The moisture content measured by Karl Fischer titration for F2 was 5.8% and for F3 4.3%. The rotavirus titer losses observed for the process from the wet film to the dried film and during storage are given in Table 17 showing relatively low values.

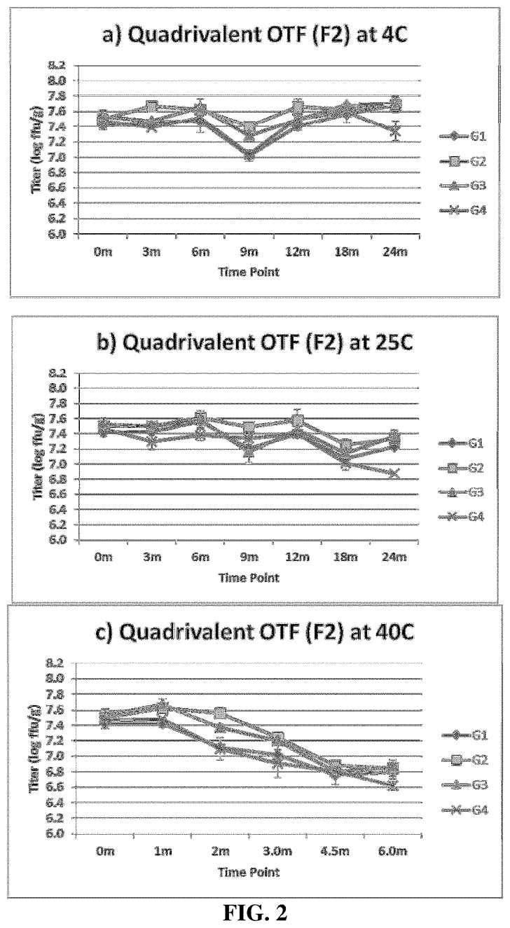

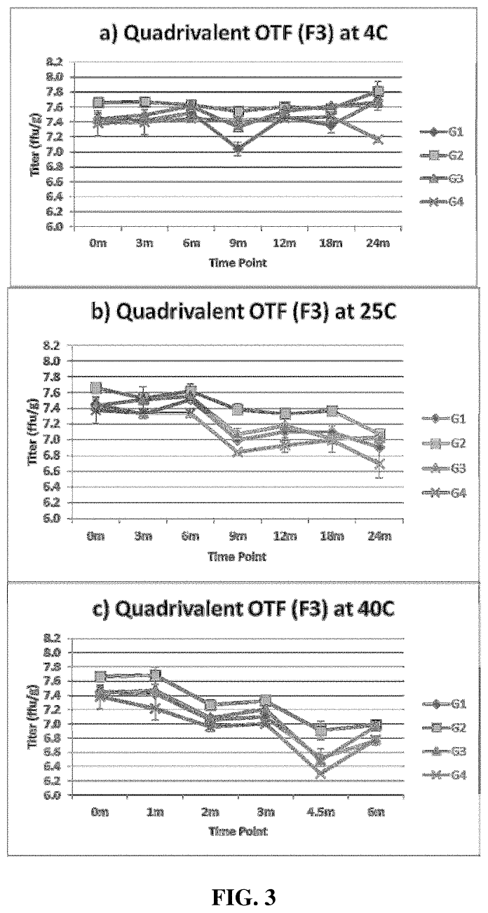

The results of the 24-month storage stability study indicated excellent storage stability across all temperatures for both formulations (FIGS. 2 and 3). After 24 months there were negligible losses in titer at 4° C. for both formulations. At 25° C., the losses after 24 months were less than 1 log, ranging from 0.2 to 0.7 log, with the G4 strain being least stable. At 40° C., the losses after 6 months for both formulations were less than 1 log also, ranging from 0.5 to 0.9, which demonstrates exceptional high temperature stability. The loss rates associated with this study are tabulated in Table 17.

TABLE 17
Results of a 24-month stability of OTF formulations
containing quadrivalent strains of rotavirus vaccine
following drying in a convection oven

| | Process Loss | | Storage stability (log ffu loss/Month) | | | | | |
|---|---|---|---|---|---|---|---|---|
| Vaccine | (log ffu) | | 4° C. | | 25° C. | | 40° C. | |
| Strain | F2 | F3 | F2 | F3 | F2 | F3 | F2 | F3 |
| G1 | 0.20 | 0.20 | −0.01 | −0.01 | 0.01 | 0.02 | 0.12 | 0.17 |
| G2 | 0.12 | 0.15 | −0.01 | 0.01 | 0.01 | 0.02 | 0.14 | 0.17 |
| G3 | 0.12 | 0.14 | −0.01 | −0.01 | 0.01 | 0.03 | 0.15 | 0.16 |
| G4 | 0.06 | 0.01 | −0.01 | 0.01 | 0.02 | 0.03 | 0.15 | 0.17 |

### Example 19—The Impact of Residual Moisture Content on the Molecular Mobility Within Films

PVA-based films (with polymer mixture T1′; produced as described in Examples 13-16) were tested using a High Flux Backscattering Spectrometer (HFBS) (conducted at NIST in Gaithersburg, Mass.) to evaluate the molecular mobility of the film matrix, with focus on the local motion (or fast dynamics). Here, the measure is the mean square amplitude of atomic motions <μ²>. These results are given in Table 18.

TABLE 18
Evaluation of molecular mobility in
OTF's containing rotavirus vaccine.

| | | Storage | |
|---|---|---|---|
| | | Stability | |
| | Moisture | @45° C. | <μ²> |
| Excipient | Content | (ffu | @45° C. |
| Profile | (wt %) | loss/wk) | (Å²) |
| F2 (0% sorbitol) | 2.7 | 0.04 | 0.28 |
| F2 (0% sorbitol) | 5.9 | 0.06 | 0.36 |
| F1 (1.6% sorbitol) | 2.6 | 0.02 | 0.30 |
| F1 (1.6% sorbitol) | 5.0 | 0.06 | 0.28 |
| F3 (5% sorbitol) | 3.1 | 0.07 | 0.29 |
| F3 (5% sorbitol) | 5.2 | 0.07 | 0.36 |
| F3 (5% sorbitol) | 7.3 | 0.25 | 0.41 |
| F8 (13% sorbitol) | 2.6 | 0.08 | 0.32 |
| F8 (13% sorbitol) | 4.4 | 0.10 | 0.35 |

The results indicate molecular mobility associated with local motion is impacted by moisture content, and, with the exception of formulation F1, higher moisture provides greater local mobility. In general the local motion magnitude was correlated with the films storage stability as well. Formulation F1, with its intermediate level of sorbitol, had molecular mobility less affected by moisture content while providing the best overall storage stability.

### Example 20A—Excipient Screening of Films with Dispersed Solid Antacid

In this example the film fabrication methods developed above for rotavirus were modified to include the incorporation of a solid dispersed antacid. The solid antacid-containing films were fabricated with limited excipient content as provided in Table 19A to evaluate the impact on process loss and storage stability of several individual buffer systems and stabilizers.

Aqueous excipient stock solutions were aseptically formulated with the excipient profiles listed in Table 19A, as described in Example 9 with pH adjusted to 6.5 with 10N KOH, but withholding the rotavirus vaccine bulk addition; an equal volume of the polymer mixture P1 was also prepared. These excipient stock solutions were first aseptically combined with CaCO₃ powder (Scoralite LL250, Scora S.A., average particle size 25 micron) to target a 25.0 wt % loading in the final film wet blend mixing on a magnetic stir plate at an approximately speed of 100 rpm for 10 minutes, which dispersed the powder evenly. Then the polymer mixture P1 was aseptically added and mixing was continued for another 5 minutes until homogenous. Lastly, the bulk rotavirus vaccine was aseptically added to the mixture and gently stirred at a speed of 80 rpm for additional 5 minutes. The film wet blend was degassed by letting it sit at room temperature for 5-10 minutes.

The resulting rotavirus film wet blend was cast on a polyethylene terephthalate (PET) backing liner (Kinmar PET, K-Mac Plastic) using a manual applicator (BYK-Gardner) at a depth of 30 mil. The wet films were dried for 90 to 120 minutes at 60° C. in a convection oven (VWR, model 1350FM).

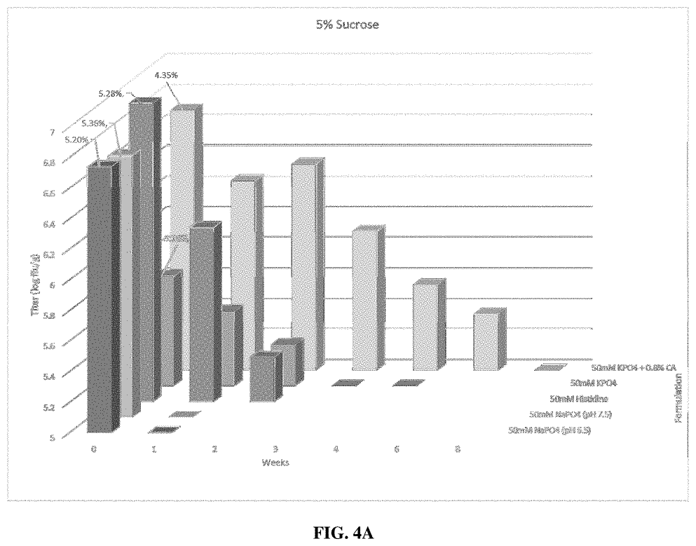

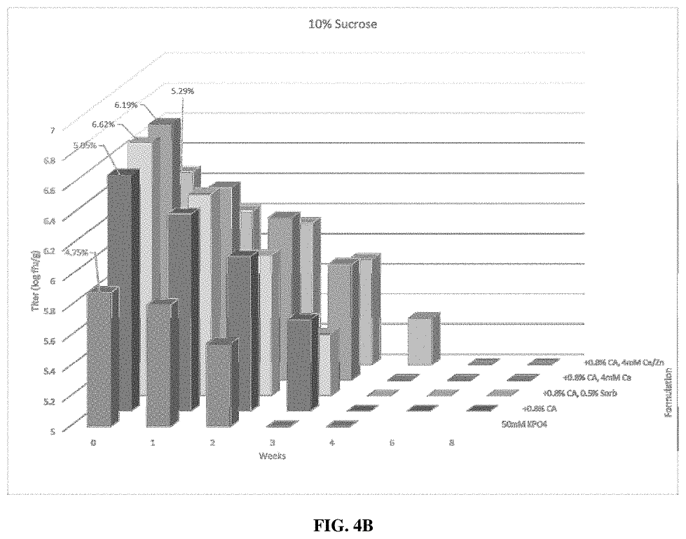

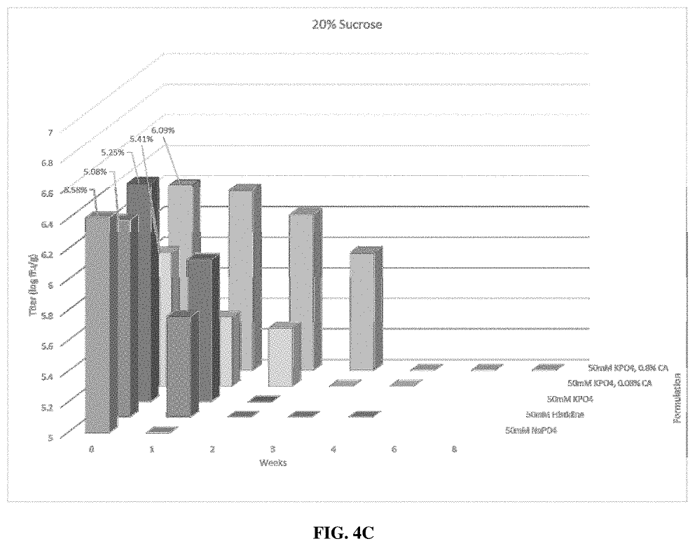

The dried films were sectioned into approximately 160 mg portions for an 8-week accelerated stability study at 45° C. The titers were determined by the FFA assay method described in Example 1. The films produced were flexible and smooth without depressions. The moisture content (measured by Karl Fischer titration and expressed on a CaCO₃-free basis), film thickness, process loss in titer to fabricate the dried film and the rotavirus titer in the OTF stability samples over the weeks at 45° C. following processing are provided in Table 19B. The storage stability data are presented in FIGS. 4 A/B/C for different sucrose content levels in the starting excipient stock solution.

TABLE 19A
Excipient profile of antacid-containing films

| Excipient | | | | | Citric | | | |
|---|---|---|---|---|---|---|---|---|
| Profile | Sucrose | | | | Acid | | | Sorbitol |
| Designation | (wt %) | Na₂PO₄ | Histidine | K₂PO₄ | (wt %) | CaCl₂ | ZnCl₂ | (wt %) |
| T3f4 | 0% | 50 mM | | | | | | |
| T3f5 | 5% | 50 mM | | | | | | |
| T3f6 | 20% | 50 mM | | | | | | |
| T3f7* | 5% | 50 mM | | | | | | |
| T3f8 | 20% | | | 50 mM | | | | |
| T3f9 | 5% | | 50 mM | | | | | |
| T3f10 | 20% | | | 50 mM | 0.08% | | | |
| T3f11 | 5% | | | 50 mM | | | | |
| T3f12 | 10% | | | 50 mM | | | | |
| T3f13 | 15% | | | 50 mM | | | | |
| T3f14 | 25% | | | 50 mM | | | | |
| T3f15 | 20% | | 50 mM | | | | | |
| T3f16 | 5% | | | 50 mM | 0.8% | | | |
| T3f17 | 10% | | | 50 mM | 0.8% | | | |
| T3f18 | 15% | | | 50 mM | 0.8% | | | |
| T3f19 | 10% | | | 50 mM | 0.8% | 4 mM | | |
| T3f20 | 10% | | | 50 mM | 0.8% | 4 mM | 4 mM | |
| T3f21 | 10% | | | 50 mM | 0.8% | | | 0.5% |
| T3f22 | 20% | | | 50 mM | 0.8% | | | |

*pH 7.5 instead of 6.5

TABLE 19B
Physical properties and stability of an OTF formulation containing rotavirus vaccine and
CaCO₃ powder following drying in a convection oven. Note that titers are expressed on a per
gram of OTF basis and the limit of quantitation for the titer assay is 5.1-5.2 log ffu/g.

| | | | | | | | | | | | | | Titer |
|---|---|---|---|---|---|---|---|---|---|---|---|---|---|
| | Drying | | | Target | | | | | | | | | loss |
| | time | | | titer of | t0 | Week 1 | Week 2 | Week 3 | Week 4 | Week 6 | Week 8 | Process | rate |
| | at 60° | Moisture | Film | film | titer | titer | titer | titer | titer | titer | titer | Loss | (log |
| Form. | C. | content | thickness | (log | (log | (log | (log | (log | (log | (log | (log | (log | ffu/ |
| # | (hour) | (%)* | (μm) | ffu/g) | ffu/g) | ffu/g) | ffu/g) | ffu/g) | ffu/g) | ffu/g) | ffu/g) | ffu/g) | wk)** |
| T3f4 | 1.5 | 4.89 | 162 | 7.2 | 6.5 | <5.2 | | | | | | −0.7 | n/a |
| T3f5 | 1.5 | 5.20 | 167 | 7.1 | 6.7 | <5.2 | | | | | | −0.4 | n/a |
| T3f6 | 2.0 | 5.58 | 150 | 7.1 | 6.4 | <5.2 | | | | | | −0.7 | n/a |
| T3f7 | 1.5 | 5.36 | 156 | 7.1 | 6.7 | <5.2 | | | | | | −0.4 | n/a |
| T3f8 | 2.0 | 5.25 | 153 | 7.1 | 6.4 | 5.9 | <5.2 | | | | | −0.7 | 0.50 |
| T3f9 | 1.5 | 5.28 | 156 | 7.2 | 7.0 | 6.1 | 5.3 | | | | | −0.2 | 0.83 |
| T3f10 | 2.0 | 5.41 | 154 | 6.9 | 5.9 | 5.5 | 5.4 | <5.1 | <5.1 | | | −1.0 | 0.25 |
| T3f11 | 1.5 | 5.16 | 153 | 6.9 | 5.7 | 5.5 | 5.3 | <5.2 | <5.2 | | | −1.2 | 0.23 |
| T3f12 | 1.5 | 4.75 | 154 | 6.9 | 5.9 | 5.8 | 5.6 | <5.2 | <5.2 | | | −1.0 | 0.17 |
| T3f13 | 1.5 | 4.50 | 156 | 6.9 | 6.0 | 5.7 | 5.6 | 5.5 | <5.1 | | | −0.9 | 0.21 |
| T3f14 | 2.0 | 5.27 | 152 | 6.9 | 6.0 | 5.3 | <5.1 | <5.1 | <5.1 | | | −0.9 | 0.65 |
| T3f15 | 2.0 | 5.08 | 156 | 6.9 | 6.3 | 5.7 | <5.1 | <5.1 | <5.1 | | | −0.6 | 0.63 |
| T3f16 | 1.5 | 4.35 | 156 | 6.9 | 6.7 | 6.2 | 6.5 | 5.9 | 5.6 | 5.4 | <5.2 | −0.2 | 0.22 |
| T3f17 | 1.5 | 5.95 | 154 | 6.9 | 6.6 | 6.3 | 6.0 | 5.6 | <5.2 | <5.2 | <5.2 | −0.3 | 0.32 |
| T3f18 | 1.5 | 5.78 | 155 | 6.9 | 6.6 | 6.3 | 6.0 | 5.6 | <5.2 | <5.2 | <5.2 | −0.3 | 0.32 |
| T3f19 | 1.5 | 6.19 | 158 | 6.9 | 6.7 | 6.3 | 6.1 | 5.8 | <5.2 | <5.2 | <5.2 | −0.2 | 0.30 |
| T3f20 | 1.5 | 5.29 | 156 | 6.9 | 6.3 | 6.0 | 5.9 | 5.7 | 5.3 | <5.2 | <5.2 | −0.6 | 0.23 |
| T3f21 | 1.5 | 6.62 | 154 | 6.9 | 6.7 | 6.3 | 5.9 | 5.4 | <5.2 | <5.2 | <5.2 | −0.2 | 0.42 |
| T3f22 | 2.0 | 6.09 | 154 | 6.9 | 6.2 | 6.2 | 6.0 | 5.8 | <5.2 | <5.2 | <5.2 | −0.7 | 0.15 |

*Expressed on a CaCO3-free basis
**Slope of the best fit line to the storage stability data.

The results indicate process loss was lowest in the histidine-buffered formulations (T3f9 and T3f15: 0.2-0.6 log ffu/g loss) and the KPO₄/0.8% citrate-buffered formulations (T3f16-T3f22: 0.2-0.7 log ffu/g loss); the NaPO₄-buffered formulations had intermediate loss (T3f4-T3f7: 0.4-0.7 log ffu/g); the KPO₄-only- and KPO₄/0.08% citrate-buffered formulations had the highest loss (T3f8, T3f10-T3f14: 0.7-1.2 log ffu/g), demonstrating the benefit of 0.8% citric acid in reducing process loss. For the KPO₄/0.8% citrate, NaPO₄, and histidine formulations greater process loss was associated with those containing 20% sucrose possibly because of the longer drying time (2 versus 1.5 hours). In contrast, the KPO₄-only formulations had lower process loss with higher sucrose content despite the longer drying time.

Factoring in the already identified differences in process loss, results in FIG. 4A illustrate the ranking in the buffers in terms of their enhancement in storage stability from best to worst as: KPO₄/Citrate, KPO4>Histidine>NaPO4. The results shown in FIG. 4B indicate improved storage stability with the combined presence of Zinc and Calcium ions, and while the addition of sorbitol is also likely stabilizing, the high moisture content of T3f21 was a confounding factor in lowering the stability of this formulation. FIG. 4C results show a similar buffer ranking as that from FIG. 4A, however, at this level of sucrose citrate provides a clearer enhancement in storage stability.

Thus the results indicate that the KPO₄/0.8% citrate buffer provides the best balance of low process loss and good storage stability for the rotavirus in an OTF formulation containing a CaCO₃ dispersed solid antacid. Zinc, calcium and sorbitol also can serve as stabilizers in these formulations, with lower moisture content further enhancing storage stability.

### Example 20 B: Films with Dispersed Solid Antacid

Building on Example 20A showing stability of solid antacid-containing films with limited excipient content, films were subsequently fabricated with a full complement of excipients including gelatin and the buffer system identified in Example 20A. The films were evaluated for storage stability and process loss for different moisture content levels. The detailed method is provided below:

Aqueous excipient stock solutions were aseptically formulated with the gelatin-containing formulations F1 and F3 (see Table 1) as described in Example 9, but withholding the rotavirus vaccine bulk addition. These excipient stock solutions were aseptically mixed in a 1:1 ratio (as if the virus bulk was included) with polymer mixture P1 on a magnetic stir plate at a speed of 100 rpm for 10 minutes. Then CaCO₃ powder (Specialty Minerals CalEssence® 1500 PCC) was aseptically added to target a 21.1 wt % loading in the final film wet blend and mixing was continued for another 5 minutes until homogenous. Lastly, the bulk rotavirus vaccine was aseptically added to the mixture and gently stirred at a speed of 80 rpm for additional 5 minutes. The film wet blend was degassed by letting it sit at room temperature for 5-10 minutes.

The resulting rotavirus film wet blend was cast on a polyethylene terephthalate (PET) backing liner (Kinmar PET, K-Mac Plastic) using a manual applicator (BYK-Gardner) at a depth of 50 mil. The wet films were dried for 120 to 180 minutes at 60° C. in a convection oven (VWR, model 1350FM).

The dried films were sectioned into approximately 160 mg portions for a 12 week accelerated stability study at 45° C. The titers were determined by the FFA assay method described in Example 1. The films produced were flexible and smooth without depressions. The moisture content (measured by Karl Fischer titration), film thickness, titer loss observed for the process to produce the dried film, and the rotavirus stability are provided in Table 19C.

TABLE 19C
Physical properties and stability of an OTF formulation
containing rotavirus vaccine and CaCO3 powder
following drying in a convection oven

| | | | | Dried Film | |
|---|---|---|---|---|---|
| | | | | Process Loss | Storage |
| Profile of | Drying | | | compared to | Stabil- |
| Excipient | time at | Moisture | Film | target titer | ity@45° C. |
| stabiliz- | 60° C. | content | Thickness | (log | (log ffu |
| ers* | (hour) | (%)** | (μm) | ffu/g)*** | loss/wk) |
| F1 | 3.0 | 5.4 | 190 | −0.1 | 0.086 |
| F1 | 2.0 | 7.0 | 235 | 0.0 | 0.299 |
| F3 | 3.0 | 4.5 | 203 | −0.3 | 0.061 |

*See Table 1.
**expressed on a CaCO₃-free basis
***expressed on a per gram of OTF basis

Relative to the OTF's produced in Example 20A (formulations), the results here show that storage stability is significantly enhanced with the addition of gelatin and that storage stability is sensitive to moisture content for films with this high loading of CaCO₃ powder, with a significant loss in stability at 7%.

### Example 21—OTFs with Monoclonal Antibody Bioactive Agents

Following similar process procedures as above for rotavirus vaccine, monoclonal-antibody-containing OTF's were prepared using heated convective drying and evaluated for loss in monomer content. Formulations with different pharmaceutical excipient stabilizers and film-forming polymers were tested for their ability to stabilize the antibody through film processing and storage at 37° C. The preparation methods are described below:

Aqueous solutions of a human IgG1 monoclonal antibody (mAb) were aseptically formulated with the different profiles of pharmaceutical stabilizers listed in Table 20 and pH adjusted to 6.5. In another container, polymer mixtures were prepared with compositions either P1 or P3. Then the formulated mAb was added to the polymer mixture in a ratio indicated in Table 20A. The film wet blends were degassed by centrifugation at a speed of 1000 rcf for 2 minutes.

The film wet blends were cast on polyethylene terephthalate (PET) backing liners (Kinmar PET, K-Mac Plastic) using manual applicators (BYK-Gardner) at different depths. The wet films were dried at 60° C. in a convection oven (VWR, model 1350FM).

The dried film was reconstituted and gently stirred to homogenize the film completely. Then the monomer content of the reconstituted mAb thin film formulation was evaluated by HPLC-SEC (high performance liquid chromatography-size exclusion chromatography). The moisture content, process loss from wet blend to film fabrication, and the 12-16 week 37° C. storage stability are provided in Table 20A. Here, the storage stability was measured by the slope of the best fit line (determined by a standard least squares statistical analysis) from a plot of % monomer content versus time.

TABLE 20
Composition of formulated antibody

| | | | Poloxamer | | | Polysorbate | |
|---|---|---|---|---|---|---|---|
| | Sorbitol | PEG400 | 188 | Sucrose | Histidine | 20 | mAb |
| Formulation | (wt %) | (wt %) | (wt %) | (wt %) | (mM) | (wt %) | (wt %) |
| M1 | | 8 | | 10 | 10 | 0.05 | 2.5 |
| M2 | | | | | 10 | 0.005 | 2.5 |
| M3 | 2.5 | | | 25 | 10 | 0.08 | 3.9 |
| M4 | | 2.5 | | 25 | 10 | 0.08 | 3.9 |
| M5 | 2.5 | | 0.08 | 25 | 10 | 0.005 | 3.9 |
| M6 | 2.4 | | | 20 | 10 | 0.005 | 3.0 |
| M7 | 2.4 | | | 8 | 10 | 0.005 | 3.0 |

TABLE 20A
Antibody film properties and stability results

| | Polymer | | | | | | Storage |
|---|---|---|---|---|---|---|---|
| | Mixture/ | | | | | | Stability |
| | Polymer:Antibody | Casting | Drying | | | | at 37° C. |
| Antibody | Mixture | Depth | Time | Thickness | Moisture | Process loss | (% monomer |
| Formulation | Ratio | (mil) | (min) | (microns) | (wt %) | (% monomer) | loss/wk) |
| M1 | P3/50:50 | 25 | 30 | 95 | 7.07 | 0.1 | 0.9 |
| M2 | P1/65:35 | 30 | 60 | 73 | 3.34 | 2.8 | 0.3 |
| M3 | P1/65:35 | 20 | 60 | 74 | 5.67 | 1.5 | 0.2 |
| M4 | P1/65:35 | 20 | 60 | 74 | 4.46 | 1.5 | 0.5 |
| M5 | P1/65:35 | 20 | 60 | 73 | 4.48 | 1.2 | 0.2 |
| M6 | P1/50:50 | 25 | 60 | 92 | 5.03 | 0.2 | 0.4 |
| M7 | P1/50:50 | 30 | 60 | 110 | 5.84 | 0.5 | 0.4 |

These findings indicate that with this film preparation method the best balance of low process loss and storage stability is provided by the sorbitol-containing formulations with the P1 polymer. These formulations are all gelatin-free.

Example 22—OTFs with Bacterial Bioactive Agent

In this example heated convective drying was used to prepare an OTF containing a live bacterial vaccine both with and without excipient stabilizers to evaluate the impact on process losses in potency. Details of the method are provided below:

Live attenuated Salmonella typhi ‘Ty21a’ vaccine was aseptically formulated in the formulation indicated in Table 21 at a titer of 7.4 log ffu/mL. T1 formulation was composed of 25 mM potassium phosphate at pH 8. T2 formulation was composed of 25% trehalose, 1% methionine, 5% gelatin, and 25 mM potassium phosphate at pH 8. In another container, the polymer mixture was prepared as described in Example 2 with 3.37% solids content. Then 8 parts of the formulated Ty21a vaccine (either T1 or T2) was added to 19 parts of the polymer mixture. This solution was dispensed into a circular dish and dried for 3 hours by convective air flow at 50° C. using a Duracraft ceramic heat furnace.

The dried film was reconstituted with sterile, filtered water to the appropriate volume and gently stirred to homogenize the film completely. Dilutions of the reconstituted Ty21avaccine were plated out onto tryptic soy agar plates warmed to room temperature. The plates were incubated at 37° C. for 20 h, and the number of colonies counted.

TABLE 21
Ty21a-containting OTF process losses for different formulations

| Formulation | Ty21a Titer Loss |
|---|---|
| # | (log ffu) |
| T1 | 2.77 |
| T2 | 0.57 ± 0.131 |

These results show the substantial benefit of a complete excipient profile of pharmaceutical stabilizers for reducing the process loss from film fabrication of an OTF containing a live bacterial vaccine.

### Example 23—OTFs with Influenza Virus

Following a similar approach to the above film fabrication methods, heated convective drying was used to produce OTF's containing live attenuated influenza vaccine. Details of the methods are provided below:

Live attenuated H1N1 influenza vaccine was aseptically formulated in Z1 formulation containing 7% sucrose and 50 mM potassium phosphate at pH 7.2 to a titer of 6.0 log ffu/mL. Similarly this vaccine was formulated in a second formulation Z2 containing 6% sucrose, 2% gelatin, 4 mM zinc chloride, 4 mM calcium chloride, 0.8% citric acid, and 50 mM potassium phosphate at pH 7.2. In a separate container, the polymer mixture was aseptically prepared as described in Example 2 with 3.37% solids content. Then 8 parts of the formulated influenza vaccine (either Z1 or Z2) was added to 19 parts of the polymer mixture. This solution was dispensed into a circular dish and dried for 3 hours by convective air flow at 50° C. using a Duracraft ceramic heat furnace.

The dried film was reconstituted with sterile, filtered water to the appropriate volume and gently stirred to homogenize the film completely. A 50% Tissue Culture Infective Dose (TCID₅₀) analysis was performed to examine titers.

### Example 24—Production of Spray Dried Powder Bioactive Agent

A spray drying process for converting the formulated liquid rotavirus vaccine into a solid state powder was developed. The detailed method and process conditions for producing four formulations were as follows:

Live monovalent rotavirus vaccine was aseptically formulated to a titer of 7.82 log ffu/mL in an aqueous wet blend with pharmaceutical stabilizers such that the resulting excipient content was as given in Table 22, with the final pH adjusted to 6.2-6.5 with 1 N KOH.

Using a Buchi Mini Spray Dryer B-290 surrounded by an environmental control chamber (ECC) and equipped with a sonic vibrating, low-pressure, 2-fluid nozzle, was fed to one port of the nozzle at 2.0 mL/min and dry nitrogen gas to the other nozzle port at 3 L/min. The inlet gas temperature to the nozzle was controlled at 86° C. and the outlet gas temperature from the drying chamber at 60° C. The temperature inside the ECC was maintained from 27.5° C. to 29.5° C. with liquid nitrogen fed directly to the ECC. The vaccine powder was collected inside the ECC by transferring to a 20 ml sterile serum glass vial. The dried powder was divided into approximately 10 mg portions for a 12-week accelerated stability study at 45° C. The powder samples were reconstituted in Trypsin-free media to determine the titer by FFA according to Example 1. The moisture content of the spray dried (SD) powder by Karl Fischer titration, the titer loss observed for the process from the wet blend to the SD powder, and the rotavirus stability are provided in Table 22.

The results demonstrated that the spray dry process can produce powders with moisture content of 2-3% while providing minimal process loss and excellent high-temperature storage stability.

TABLE 22
Spray Dried (SD) Wet Blend Formulation Compositions

| | | | | | Zn | Ca | | |
|---|---|---|---|---|---|---|---|---|
| SD Wet | Trehalose | Sucrose | Glycerin | Histidine | Cl | Cl | Serine | Methionine |
| Blend | % w/v | % w/v | % w/v | mM | mM | mM | % w/v | mM |
| SD1 | 19.0 | | 0.40 | 20 | 3 | 3 | 0.50 | 10 |
| SD2 | | 17.0 | | | 3 | | | 10 |
| SD3 | 17.0 | | | | 3 | | | 10 |
| SD4 | 19.0 | | | 20 | 3 | 3 | | |

| | | | | | | | Storage |
|---|---|---|---|---|---|---|---|
| | | | | | Powder | Process | Stability |
| | | | | Poloxamer | Moisture | Loss | @45° C. |
| SD Wet | KPO4 | Citrate | Gelatin | 188 | Content | (log | (log ffu |
| Blend | mM | mM | % w/v | % w/v | (%) | ffu) | loss/wk) |
| SD1 | | | | | 2.0 | 0.0 | 0.00* |
| SD2 | 20 | 20 | 2.0 | 0.008 | 2.6 | 0.0 | 0.015 |
| SD3 | 20 | 20 | 2.0 | 0.008 | Not | Not | Not |
| | | | | | measured | measured | measured |
| SD4 | | | | | 2.4 | Not | Not |
| | | | | | | measured | measured |

*No measureable loss after 12 weeks storage.

### Example 25—OTFs Using Organic Solvents and Spray Dried Bioactive Agent

In this example a method of preparation of OTF's containing the spray dried live rotavirus vaccine powders produced in Example 24 was developed. Two different organic solvents were used and the storage stability of the resulting films was evaluated. The detailed methods are below.

The SD powders from Example 24 were aseptically mixed with an organic solvent containing dissolved water-soluble polymers such that the composition of the resulting liquid mixture (film wet blend) was 4% by weight SD powder, 15% PVP (Kollidon 90 F), 1.7% PEG 400 (polyethylene glycol, Mw-400), and 79.3% organic solvent as indicated in Table 23. The film wet blend was mixed on a stir plate at 100 rpm for about 2 minutes until homogenous prior to casting.

The films were case on a fluoropolymer coated polyester backing liner (3M Scotchpak 1022) using a manual applicator (BYK-Gardiner) at a thickness of 30 ml. The wet films were dried for 3 hours at 40° C. in a convection oven (VWR, model 1350FM). The moisture content of the dried films was determined by Karl Fisher titration (Table 23). The dried films were sectioned into approximately 100 mg portions for an accelerated stability study at 45° C. The film samples were reconstituted in trypsin-free media to determine the titer of by FFA according to Example 1. The films were flexible and smooth without depressions. The results of the rotavirus process loss observed for the process through spray drying and film casting and drying, and the storage stability are shown in Table 23.

The results indicate some dependence of the solvent on the final moisture content (ethanol formulations are more dry than those using isopropanol), although there does not appear to be a discernable impact of moisture on stability in this range of 1.1 to 2.3%. There was low process loss for the one batch that it was measured. Storage stability at 45° C. for the films containing one spray dried powder formulation (SD1) being exceptional.

TABLE 23
Film formulations prepared containing spray dried rotavirus vaccine-containing powders

| | | | | | | | |
|---|---|---|---|---|---|---|---|
| Film | SDF1 | SDF2 | SDF3 | SDF4 | SDF5 | SDF6 | SDF7 |
| formulation | | | | | | | |
| SD powder | SD1 | SD1 | SD2 | SD3 | SD3 | SD4 | SD4 |
| Solvent | Ethanol | Isopropanol | Isopropanol | Ethanol | Isopropanol | Ethanol | Isopropanol |
| Film Moisture | 1.1 | 2.0 | 1.6 | 1.7 | 2.3 | 1.6 | 2.2 |
| Content % | | | | | | | |
| Dry film | 121 | Not | 107 | Not | Not | 109 | Not |
| thickness (μm) | | measured | | measured | measured | | measured |
| Process Loss | 0.28 | 0.25 | 0.0 | 0.0 | 0.0 | 0.0 | 0.14 |
| (log ffu) | | | | | | | |
| Storage | 0.028 | 0.004 | 0.094 | 0.091 | 0.112 | 0.132 | 0.103 |
| stability@45° C. | | | | | | | |
| (log ffu | | | | | | | |
| loss/wk) | | | | | | | |

### Example 26—Immunogenicity study of OTF formulation in mice

An immunogenicity study of rhesus rotavirus vaccine (RRV) oral dosage presentations in 7-day old BALB/c mouse pups was performed using liquid and OTF formulations to compare their ability to elicit an immune response. The RRV vaccine (obtained from Professor Harry Greenberg's lab, Stanford University) was used because it is known to be significantly more immunogenic in mice than the human-bovine rotavirus vaccines used in the prior examples. The methods are described below:

The live RRV OTF was aseptically formulated to a titer of 6.3 log pfu/dose with F1 formulation composition (see Table 1) as described in Example 13. Here, a dose consisted of two 3 mm diameter film discs and were placed inside the cheek of the mouse pups. Liquid formulations consisted of reconstituted film and bulk unformulated RRV also 6.3 log pfu/dose, with dose volume of 100 uL/dose delivered by oral gavage. Saline was also dosed to a mouse group to serve as a control.

For each of the four groups of mice (5 mice/group), three dosings occurred at 2 week intervals. Stool and serum samples were also collected at 2 week intervals to measure anti-RRV IgA and IgG antibody response, respectively, by ELISA assay. Mouse pups were 7 days old on the day of the first dosing.

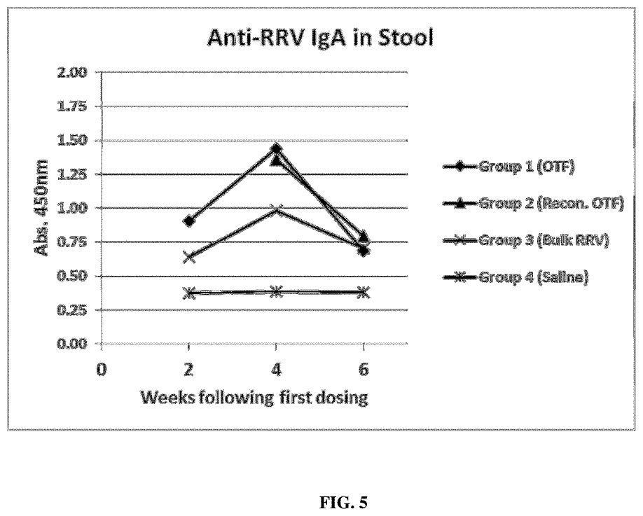

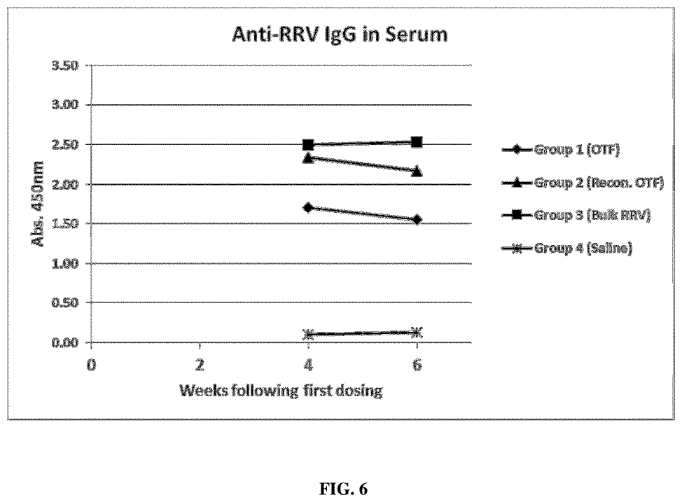

The results for the stool IgA and serum IgG response are provided in FIGS. 5 and 6. Although stool IgA response was greatest for the OTF and reconstituted OTF, while the serum IgG response was greatest for the bulk RRV, all were significantly greater than the saline group, indicating comparably good immune response from both OTF and bulk RRV, which demonstrates immunogenicity is not impaired by the OTF preparation method or the formulation components.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A quick-dissolving thin film composition comprising:
one or more matrix polymers;
a bioactive agent;
one or more pharmaceutically acceptable excipients; and,
a buffer.

2-3. (canceled)

4. The quick dissolving thin film composition of claim 1, wherein the one or more polymers comprise a two-polymer composition which includes polyvinyl pyrrolidone with polyethylene oxide or polyvinyl pyrrolidone with polyethylene glycol.

5-6. (canceled)

7. The quick dissolving thin film composition of claim 1, wherein the bioactive agent is a protein, an antibody, or a vaccine.

8-13. (canceled)

14. The quick dissolving thin film composition of claim 1, wherein the pharmaceutically acceptable excipients are selected from the group consisting of a polyol, metal ions, antacid, amino acid, protein, sugars, carboxylate, surfactants, gelatin and combinations thereof.

15. The thin film of claim 1, wherein the excipients comprise Zn²⁺ and Ca²⁺.

16-19. (canceled)

20. The quick dissolving thin film composition of claim 1 wherein the buffer is selected from the group consisting of: histidine, HEPES, Tris, acetate, citrate, succinate, tartarate, maleate, lactate, ammonium bicarbonate, phosphate, magnesium oxide, aluminum oxide, aluminum hydroxide with magnesium hydroxide, aluminum carbonate gel, calcium carbonate, sodium bicarbonate, hydrotalcite, sucralfate, bismuth subsalicylate, and combinations thereof.

21-22. (canceled)

23. The quick dissolving thin film composition of claim 14, wherein the pharmaceutically acceptable excipients in the film comprise: polyol at a concentration ranging from 5% to about 70% (w/w); a carboxylate ranging in concentration from about 0.02 mmol/g to about 1 mmol/g; Zn²⁺ in a concentration ranging from about 0.0015 mmol/g to 0.075 mmol/g; and wherein the buffer is a phosphate buffer ranging in concentration from about 0.01 mmol/g to about 3 mmol/g.

24-27. (canceled)

28. The quick dissolving thin film composition according to claim 14, wherein the pharmaceutically acceptable excipients are comprising polyol at a concentration between about 35% to about 45%; at least one carboxylate at a concentration between about 0.05 mmol/g and about 0.1 mmol/g; Ca²⁺ in a concentration ranging from about 0.005 mmol/g to 0.01 mmol/g; Zn²⁺ in a concentration ranging from about 0.005 mmol/g to 0.01 mmol/g; gelatin in a concentration ranging from about 5% to about 10%; and wherein the phosphate buffer is phosphate at a concentration between about 0.05 to about 0.15 mmol/g.

29. The quick dissolving thin film composition of claim 4, wherein the composition is formed with polyvinyl pyrrolidone and polyethylene oxide or polyethylene glycol polymers first dissolved in an organic solvent and then combined with a dried bioactive agent comprising one or more pharmaceutically acceptable excipients and a buffer.

30-32. (canceled)

33. The quick dissolving thin film composition of claim 29, wherein the bioactive agent is not encapsulated in a membrane within the polymer matrix.

34. The quick dissolving thin film composition of claim 29, wherein the pharmaceutically acceptable excipients comprise: a polyol at a concentration ranging from about 5% to about 50% (w/w); a carboxylate ranging in concentration from about 0.01 mmol/g to about 1 mmol/g; Zn²⁺ in a concentration ranging from about 0.0015 to 0.075 mmol/g; and a phosphate buffer ranging in concentration from about 0.01 mmol/g to about 3 mmol/g.

35-42. (canceled)

43. The quick dissolving thin film composition of claim 1, wherein the films are multilayered laminates incorporating separate layers comprising antacids or mucoadhesives.

44-47. (canceled)

48. A method of preparing a quick-dissolving thin film composition comprising:
providing one or more polymers;
providing a bioactive agent;
providing one or more pharmaceutically acceptable excipients;
combining the bioactive agent with the excipients in a solution or suspension;
combining the solution or suspension with the one or more polymers to form a wet blend;
applying the wet blend to a flat surface; and,
drying the wet blend to form a dry thin film;
wherein the dry thin film comprises less than 5% residual moisture.

49-50. (canceled)

51. The method of claim 48, where a suitable volatile organic solvent is added to the film wet blend prior to extrusion or casting to enhance the drying efficiency.

52. The method of claim 48, whereby a solution of one or more pharmaceutically acceptable excipients and a buffer is combined with a solution of one or more polymers, followed by addition of antacid powder that is dispersed by mixing, followed by the addition of the bioactive agent dispersed by mixing, and wherein the film is formed by extrusion or casting onto a flat surface and drying said film under laminar flow, heating, vacuum, or a drying combination thereof.

53-56. (canceled)

57. The method of claim 48, wherein the pharmaceutically acceptable excipients are selected from the group consisting of: a polyol, metal ions, antacid, amino acid, protein, a plasticizer, carboxylate, surfactants, and gelatin.

58. (canceled)

59. A thin film composition of rotavirus, the composition comprising: stabilizer excipients comprising potassium phosphate, citric acid, sucrose, sorbitol, calcium chloride, zinc chloride, and gelatin; a polyvinyl alcohol; and, a rotavirus.

60-67. (canceled)

68. The thin film of claim 59, wherein the film has a major plane with a thickness ranging from 50 microns to 200 microns.

69. The thin film of claim 59, wherein the composition is essentially free of gelatin.

70. The thin film composition of claim 1, comprising:
excipients comprising sorbitol, poloxamer, sucrose, histidine, and polysorbate;
a polyvinyl alcohol matrix polymer; and,
an antibody.

71-73. (canceled)

74. The method of claim 48, wherein providing the bioactive agent in the thin dry film comprises:
blending together the bioactive agent, one or more matrix polymers, and an aqueous excipient solution to form a wet blend;
applying the wet blend to a flat surface;
drying the wet blend to form the thin dry film composition; and
wherein the bioactive agent comprises a rotavirus, the one or more matrix polymers comprises PVA, and the excipient solution comprises sorbitol, zinc cation, calcium cation.

75. (canceled)

76. The method of claim 48, wherein the bioactive agent is an antibody agent in the thin dry film, the method further comprising:
blending together the antibody bioactive agent, one or more matrix polymers, and an aqueous excipient solution to form a wet blend;
applying the wet blend to a flat surface; and,
drying the wet blend to form the thin dry film composition;
wherein the matrix polymer comprises PVA, and the excipient solution comprises histidine.

77-78. (canceled)
